SEQUENCE LISTING (3 sequences)

SEQ ID NO: 1 - protein, 17 aa, Homo sapiens
QRFCTGHFGGLYPCHGP

SEQ ID NO: 2 - protein, 11 aa, Human immunodeficiency virus
YGRKKRRQRRR

SEQ ID NO: 3 - protein, 18 aa, Homo sapiens
APWISLRGDDTGVLLPTP

(19) United States
(12) Patent Application Publication
(10) Pub. No.: US 2024/0002474 A1
(43) Pub. Date: Jan. 4, 2024

(54) Systems and Methods for Diagnostic Assessment and Treatment of Insulin Resistance and Hyperglycemia

(71) Applicant: The Board of Trustees of the Leland Stanford Junior University, Stanford, CA (US)

(72) Inventors: Michael P. Snyder, Stanford, CA (US); Andrew Lipchik, Palo Alto, CA (US); Justin Annes, Palo Alto, CA (US); Sooyeon Lee, Santa Clara, CA (US); Anil Narasimha, Stanford, CA (US)

(73) Assignee: The Board of Trustees of the Leland Stanford Junior University, Stanford, CA (US)

(21) Appl. No.: 18/253,198

(22) PCT Filed: Nov. 16, 2021
(86) PCT No.: PCT/US21/72445

§ 371 Date: May 16, 2023

Related U.S. Application Data
(60) Provisional application No. 63/114,425, filed on Nov. 16, 2020.

(51) Int. Cl. C07K 16/00; A61P 3/10; A61P 5/50

(52) U.S. Cl. CPC C07K 16/00; A61P 3/10; A61P 5/50; C07K 2317/41; C07K 2317/52

(57) ABSTRACT

Systems and methods to assess insulin resistance are described. Further, compounds and methods for treatment of insulin resistance or hyperglycemia are described. In some instances, immunoglobins of an individual are utilized in an insulin-stimulated mitochondrial function assay, which can be utilized to determine insulin resistance. In some instances, glycosylation, including sialylation, of immunoglobins of an individual are assessed, which can be utilized to assess insulin resistance. In some instances, immunoglobins, such as immunoglobin G, are utilized as a treatment for insulin resistance or hyperglycemia.

### CROSS REFERENCE TO RELATED APPLICATIONS

This application claims priority to U.S. Provisional Application Ser. No. 63/114,425 entitled “Diagnostic Assessment and Treatment of Insulin Resistance and Hyperglycemia,” filed Nov. 16, 2020, which is herein incorporated by reference in its entirety.

### STATEMENT REGARDING FEDERALLY SPONSORED RESEARCH OR DEVELOPMENT

This invention was made with Government support under contracts DK101530, DK104460 and DK102556 awarded by the National Institutes of Health. The Government has certain rights in the invention.

### SEQUENCE LISTING

The instant application contains a Sequence Listing which has been submitted electronically in ASCII format and is hereby incorporated by reference in its entirety. The ASCII copy was created on Nov. 16, 2021, is named 07043 Seq List_ST25.txt, and is 903 bytes in size.

### TECHNICAL FIELD

The disclosure is generally directed to processes to assess and treat insulin resistance, hyperglycemia, and type 2 diabetes.

### BACKGROUND

One in ten individuals are affected by diabetes, a condition involving abnormal regulation of glycemia (i.e., the level of sugar or glucose in blood). Standard assessments of glycemia typically utilize single time or average measurements of blood glucose. A few common methods to assess glycemia include measuring fasting plasma glucose (FPG), glycated hemoglobin (HbA1c test), and oral glucose tolerance test (OGTT). In addition, individuals can be tested for their insulin resistance using an insulin suppression test that characterizes the steady-state plasma glucose (SSPG).

Each glycemia assessment yields different insight. FPG is a measure of glucose levels at a steady state where production of glucose by the liver and kidney needs to match glucose uptake by tissues. Impaired FPG typically results from a mismatch between glucose production and glucose utilization. In contrast, OGTT measures a dynamic response to a glucose load which leads to increased plasma insulin which suppresses hepatic glucose release and stimulates glucose uptake in the peripheral tissues. Impaired pancreatic beta cell function and peripheral insulin resistance, particularly in skeletal muscle, can lead to impaired glucose tolerance (IGT). The ambient glucose concentration determines the rate of formation of HbA1C in erythrocytes which have a lifespan of ˜120 days. Accordingly, HbA1C reflects average blood glucose levels over the past 3-4 months.

Insulin resistance is a pathological condition in which cells fail to respond to insulin. Healthy individuals respond to insulin by using the glucose available in the blood stream and inhibit the use of fat for energy, which allows blood glucose to return to the normal range. To perform an insulin suppression test, both glucose and insulin are suppressed from an individual's bloodstream by intravenous infusion of octreotide. Then, insulin and glucose are infused into the bloodstream at a particular rate and blood glucose concentrations are measured at a number of time checkpoints to determine the ability of the individual to respond to insulin, resulting in a determination of SSPG levels. Subjects with an SSPG of 150 mg/dL or greater are considered insulin-resistant; however, this cutoff can vary depending upon the interpreter.

### SUMMARY

Several embodiments are directed towards analysis of an individual's immunoglobins (Igs), especially the glycosylation and sialyation of immunoglobin G (IgG). In many embodiments, a biological sample of an individual comprising IgG is examined, in which can determine an individual's insulin sensitivity. In several embodiments, the individual's biological sample comprising IgGs is assessed by performing a mitochondrial function assay, which can indicate insulin sensitivity. In many embodiments, the amount of sialyation on IgG is determined, which can also indicate insulin sensitivity. In some embodiments, once insulin sensitivity is determined, a clinical intervention and/or treatment is performed.

Several embodiments are directed towards utilizing immunoglobins for treatment of insulin resistance and/or diabetes. In many embodiments, IgG is utilized for treatment of insulin resistance and/or diabetes. In several embodiments, an Fc fragment of IgG is utilized for treatment of insulin resistance and/or diabetes. In many embodiments, IgG or fragments thereof are sialylated when utilized for treatment of insulin resistance and/or diabetes. In some embodiments, an individual's IgG sample is sialylated in vitro and then utilized for treatment of insulin resistance and/or diabetes.

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIG. 1 provides a schematic depicting the timeline and pathologies of insulin resistance, prediabetes and type 2 diabetes.

FIG. 2 provides a Venn diagram of the results of testing a cohort for prediabetes utilizing three standard assessments, generated in accordance with the prior art. The standard assessments are fasting plasma glucose, HbA1c levels, and oral glucose tolerance test (OGTT).

FIG. 3 provides exemplary results of sera derived from healthy, insulin resistant, and type 2 diabetic individuals in an insulin-stimulated mitochondrial function test, generated and utilized in accordance with various embodiments.

FIG. 4 provides a graph depicting the correlation between an insulin-stimulated mitochondrial function test and an insulin suppression test for insulin resistance, generated and utilized in accordance with various embodiments.

FIG. 5 provides a flow chart of a process to assess insulin-stimulated mitochondrial respiration in animal cells treated with a subject's IgGs in accordance with various embodiments.

FIG. 6 provides an exemplary method of performing an insulin-stimulated mitochondrial respiration assessment in accordance with various embodiments.

FIG. 7 provides a schematic detailing the relationship of glycosylation patterns of IgG and insulin sensitivity/resistance, utilized in accordance with various embodiments.

FIG. 8 provides a flow chart of a process to assess sialylation of a subject's IgGs in accordance with various embodiments.

FIGS. 9 and 10 provide immunoblot analysis of Akt phosphorylation in response to insulin stimulation following acute exposure of HepaRG (FIG. 9) and C2C12 myotubes (FIG. 10) to diluted (1:100) patient serum (n=3), utilized in accordance with various embodiments. Data were analyzed using a one-way analysis of variance (ANOVA) with Tukey multiple comparisons test.

FIGS. 11A to 11C provide data graphs characterizing the insulin-dependent mitochondrial respiration in response to insulin-stimulation with and without serum starvation in HepaRG hepatocytes, generated in accordance with various embodiments.

FIG. 12 provides data graphs characterizing the insulin-dependent mitochondrial respiration in human primary skeletal muscle cells and C2C12 myotubes, generated in accordance with various embodiments.

FIGS. 13 to 15 provide tables of demographic data of cohort 1 (FIG. 13), cohort 2 (FIG. 14), and a type 2 diabetic cohort (FIG. 15), utilized in accordance with various embodiments.

FIGS. 16A and 16B provide data graphs of raw mitochondrial respiration measurements in HepaRG hepatocytes following acute exposure to individual's serum, generated in accordance with various embodiments.

FIG. 17 provides a data graph of showing that an individual serum suppresses insulin-dependent mitochondrial respiration in muscle cell model, generated in accordance with various embodiments.

FIG. 18 provides data graphs comparing clinical parameters between in insulin sensitive (SSPG<150), insulin resistant (SSPG>150), and type 2 diabetic individuals, utilized in accordance with various embodiments. Data were analyzed using a one-way analysis of variance (ANOVA) with Tukey multiple comparisons test. Statistical significance is indicated as follows: *P<0.05, **P<0.01, ***P<0.001, and ****P<0.0001.

FIGS. 19A and 19B provide data graphs depicting serial dilution of insulin resistant and type 2 diabetic serum maintains suppression of insulin-dependent mitochondrial respiration, generated in accordance with various embodiments.

FIGS. 20A and 20B provide data graphs depicting serum fractions greater than 50 kDa is necessary for suppression of mitochondrial respiration, generated in accordance with various embodiments.

FIG. 21 provide mitochondrial insulin stimulation test oxygen consumption rate profile in HepaRG hepatocytes following acute exposure to purified IgG (10 μg) from insulin-sensitive (n=14), insulin resistant (n=25), or type 2 diabetic (n=12) patient serum (left panel), generated in accordance with various embodiments. Data represent the mean ±s.e.m of each cohort performed in triplicate. FIG. 21 further provides insulin-dependent and FCCP-dependent maximal respiration oxygen consumption rates, generated in accordance with various embodiments. Data were analyzed using a one-way ANOVA with Tukey multiple comparisons test.

FIG. 22 provides a data graph depicting the correlation of insulin-stimulated normalized oxygen consumption rate after purified IgG treatment with insulin sensitivity as measured by MIST, generated and utilized in accordance with various embodiments.

FIG. 23 provides a schematic of generating immunoglobin fractions Fab and Fc, utilized in accordance with various embodiments.

FIG. 24 provides data graphs depicting insulin-stimulated normalized OCR following treatment with purified Fab fragments from insulin-resistant patient serum, generated in accordance with various embodiments. FIG. 24 further provides insulin-dependent oxygen consumption rates from MIST assays pre-treated with Fab or whole IgG from IR serum (n=14), generated in accordance with various embodiments. Data were analyzed using a two-sided Student's t-test.

FIG. 25 provides mitochondrial insulin stimulation test oxygen consumption rate profile in HepaRG hepatocytes following following treatment with Fc Block reagent then acute exposure to insulin-sensitive (n=11, blue), insulin resistant (n=26, red), or type 2 diabetic (n=12, orange) patient serum for 4 hours, generated in accordance with various embodiments.

FIG. 26 provides data graphs depicting Fc receptor blockade rescues serum-dependent suppression of mitochondrial function in skeletal muscle cells, generated in accordance with various embodiments.

FIG. 27 provides immunoblots analyzing Akt phosphorylation following insulin stimulation with or without Fc Block, utilized in accordance with various embodiments. No—no serum treatment, NA—not applicable, and NP—not performed.

FIG. 28 provides immunoblots showing Fc receptor profiling and confirmation of FcRn knockdown in HepaRG hepatocytes, utilized in accordance with various embodiments.

FIG. 29 provides mitochondrial insulin stimulation test oxygen consumption rate profile in FcRn shRNA knockdown HepaRG hepatocytes following acute exposure to serum from insulin-sensitive (n=14), insulin resistant (n=25), or type 2 diabetic (n=12) patient serum, generated in accordance with various embodiments.

FIG. 30 provides a schematic showing strategies for inhibition of FcRn using Fc-binding domain inhibitor SYN746 or a cell-penetrating peptide containing the FcRn intercellular residues 330-347 N-terminal tail as a dominant-negative, utilized in accordance with various embodiments.

FIGS. 31 and 33 provide normalized OCR profiles following 10-minute pretreatment with SYN746 (10 μM) or FcRn330-447-TAT (10 μM) before exposure to diluted patient serum, generated in accordance with various embodiments. Data represent mean±s.e.m of each cohort performed in triplicate.

FIG. 32 provides data graphs depicting insulin-dependent respiration and FCCP uncoupling respiration under all FcRn targeted treatments, generated in accordance with various embodiments. Data were analyzed using a one-way ANOVA with Tukey multiple comparisons test.

FIG. 34 provides a schematic of experiment strategy to evaluate the efficacy of IgG on insulin sensitivity in genetically leptin-receptor deficient mice (db/db) and control (C57BL6) mice. ITT—insulin tolerance test, FBG—fasting blood glucose, FBI—fasting blood insulin, utilized in accordance with various embodiments.

FIG. 35 provides data graphs depicting longitudinal insulin tolerance test following IPIG in leptin receptor deficient (db/db) mice and control (C57BL6) mice, generated in accordance with various embodiments.

FIGS. 36 to 38 provide data graphs of results of single intraperitoneal dose of mouse IgG (1 g kg⁻¹) or saline (vehicle) in the fasted state of 10-week old genetically leptin-receptor deficient mice (db/db) and control (C57BL6) mice, generated in accordance with various embodiments. Insulin tolerance tests performed after a single intraperitoneal dose of saline or IgG in 10-week old control (C57BL6) (n=17) or diabetic (Lepr db/db) (n=17) mice on day 0 (FIG. 36) and 35 days later (FIG. 36, n=11-12). Data represent the mean±s.e.m. of two independent experiments (n=5-6) for control mice treated with saline (black) or IPIG (green) and db/db mice treated with saline (red) or IPIG (blue). Longitudinal ad libitum (FIGS. 37) and 4-hour fasting (FIG. 37) blood glucose levels following administration of IgG or saline. (FIG. 38) Four hour fasting glucose and insulin levels 16 days post-IPIG or saline treatment. (FIG. 38) Overnight fasting blood glucose and insulin levels 28 days following administration of IgG or saline. Data were analyzed by one-way ANOVA with Tukey's multiple comparisons test. Statistical significance is indicated as follows: *P<0.05, **P<0.01, ***P<0.001, and ****P<0.0001.

FIG. 39 provides microscopy images and data graphs of adipocyte cell size of diabetic mice treated with IgG, generated in accordance with various embodiments.

FIG. 40 provides microscopy images and data graphs of murine islets of 13 cells of diabetic mice treated with IgG, generated in accordance with various embodiments.

FIG. 41 provides a schematic and immunoblot of chimeric IgG-IgM Fc peptides, utilized in accordance with various embodiments.

FIGS. 42 and 43 provide data graphs depicting the insulin sensitivity of diabetic mice treated with IgG Fc peptides or chimeric IgG-IgM Fc peptides after administration (FIGS. 42) and 1 week after administration (FIG. 43), generated in accordance with various embodiments.

### DETAILED DESCRIPTION

Turning now to the drawings and data, systems and methods to assess and treat insulin resistance, hyperglycemia, and/or type 2 diabetes and in accordance with various embodiments are described. In some embodiments, a diagnostic assessment of a subject's glycemia is determined utilizing a biological sample of the subject inclusive of immunoglobulins (Igs), especially immunoglobulin G (IgG). In some embodiments, the biological sample containing Igs is utilized in a diagnostic assay that assesses mitochondrial respiration stimulated by insulin, the results of which are a correlative surrogate for assessing insulin sensitivity. In some embodiments, the Igs within the biological sample are assessed for glycosylation patterns, in which certain glycosylation patterns (especially sialylation) have been found to be correlative with insulin resistance. In some embodiments, an individual is diagnosed as being insulin resistant, having prediabetes, and/or having type 2 diabetes, based on the effect of an Ig sample on mitochondrial respiration and/or an Ig glycosylation assessment. Various embodiments utilize an individual's diagnostic assessment to perform further diagnostic testing and/or treat the individual for insulin resistance and/or hyperglycemia. In some embodiments, an individual is treated with Igs (especially the Fc region of Igs) to mitigate insulin resistance and/or promote insulin sensitivity. In some instances, a treatment can include a medication (e.g., metformin), a dietary supplement, dietary restrictions, physical activity, and any combination thereof.

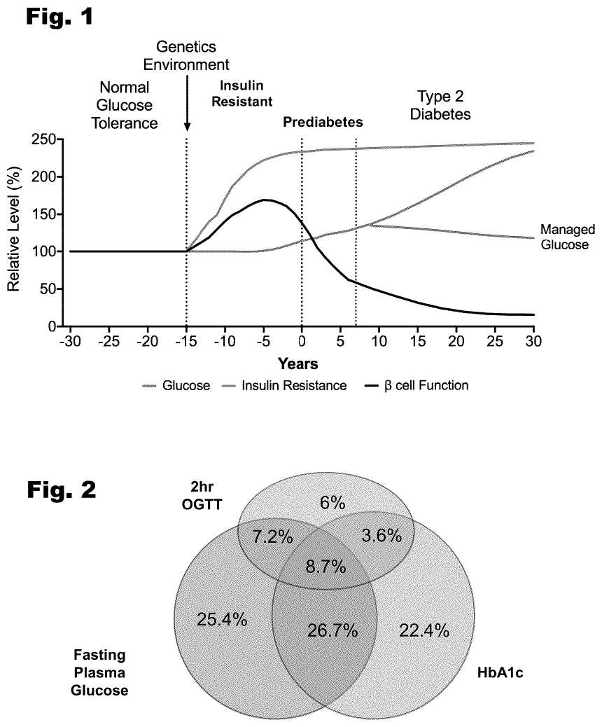

Current diagnostic tests to diagnose prediabetes and insulin resistance are inadequate. The typical pathology of type 2 diabetes is depicted in FIG. 1. Prior to development of type 2 diabetes, most individuals first unwittingly experience insulin resistance. At this initial stage, the body adapts to the insulin resistance and maintains blood glucose at normoglycemic levels by increasing β cell function and insulin production. As the pathology progresses, insulin resistance continues to increase and β cell function is unable to be maintained at elevated levels and actually begins to regress. As β cell function decreases, the body is unable to maintain glucose at normoglycemic levels and enters into a prediabetic phase characterized by slight hyperglycemia. Further loss of β cell function results in increased hyperglycemia and development of type 2 diabetes.

To prevent the onset of type 2 diabetes, it is ideal diagnose insulin resistance at its earliest stages. The insulin suppression test is currently the best test to assess insulin resistance, but the test is not performed often due to being an unpleasant, time-consuming, and resource intensive exam. Further, accurate assessment of prediabetes is difficult. Assessment of prediabetes via fasting plasma glucose (FPG), glycated hemoglobin (HbA1c test), and oral glucose tolerance test (OGTT) is incongruent. In a study performed by E. Barry et al. that compared the ability of these tests to diagnose prediabetes, only 8.7% of individuals were diagnosed as prediabetic by all three tests (FIG. 2), highlighting the fact that a certain percentage of individuals having prediabetes are misdiagnosed as healthy depending on the assay performed (E. Barry, et al., BMJ 356:i6538, 2017, the disclosure of which is incorporated herein by reference). Thus, there is a need to improve early diagnosis in the pathology of insulin resistance, hyperglycemia, and type 2 diabetes.

Various embodiments described within this disclosure are based on the discovery that an individual's immunoglobins (Igs) provide a means to diagnose insulin resistance. In some embodiments, a biological sample inclusive of Igs is collected from an individual and assessed in an insulin-stimulated mitochondrial respiration assay. In some embodiments, an individual's Igs are examined for particular glycosylation patterns indicative insulin resistance.

Further, various embodiments described within this disclosure are based on the discovery that sialylated Igs mitigate insulin resistance and increase insulin sensitivity. Accordingly, in some embodiments, Ig (especially sialylated Ig) is utilized as a treatment to promote insulin sensitivity. In some embodiments, Ig (especially sialylated Ig) is utilized as a treatment to counter insulin resistance, progression of insulin resistance, and/or development of diabetes. In some embodiments, a Fc peptide of an IgG (especially a sialylated Fc peptide) is utilized as a treatment to promote insulin sensitivity. In some embodiments, a Fc peptide of an IgG (especially a sialylated Fc peptide) is utilized as a treatment to counter insulin resistance, progression of insulin resistance, and/or development of diabetes. In some embodiments, a chimeric Fc peptide (e.g., IgG/IgM chimeric Fc peptide) is utilized to promote insulin sensitivity. In some embodiments, a chimeric Fc peptide (e.g., IgG/IgM chimeric Fc peptide) is utilized as a treatment to counter insulin resistance, progression of insulin resistance, and/or development of diabetes. In some embodiments, a chimeric Fc peptide used for treatment is sialylated. In some embodiments, a compound that increases sialylation of Igs is utilized as a treatment to promote insulin sensitivity. In some embodiments, a compound that increases sialylation of IgG is utilized as a treatment to counter insulin resistance, progression of insulin resistance, and/or development of diabetes. Compounds that increase sialylation of IgG include (but are not limited to) sialic acid precursors, an agonist of sialyltransferase or an antagonist of neuroaminidase. Sialic acid precursors include (but are not limited to) ManNAc and Neu5Ac.

In some embodiments, an individual is administered Ig (especially sialylated Ig) to promote insulin sensitivity. In some embodiments, an individual is administered an Ig (especially sialylated IgG) to promote insulin sensitivity. In some embodiments, an individual is administered a Fc peptide of an IgG (especially a sialylated Fc peptide). In some embodiments, an individual is administered a chimeric Fc peptide (e.g., IgG/IgM chimeric Fc peptide). In some embodiments, a chimeric Fc peptide used for administration is sialylated. In some embodiments, an individual is administered a compound that increases sialylation of IgG, such as (for example) sialic acid precursors, an agonist of sialyltransferase or an antagonist of neuroaminidase. Sialic acid precursors include (but are not limited to) ManNAc and Neu5Ac.

### Assessment of Insulin Resistance via Mitochondrial Function

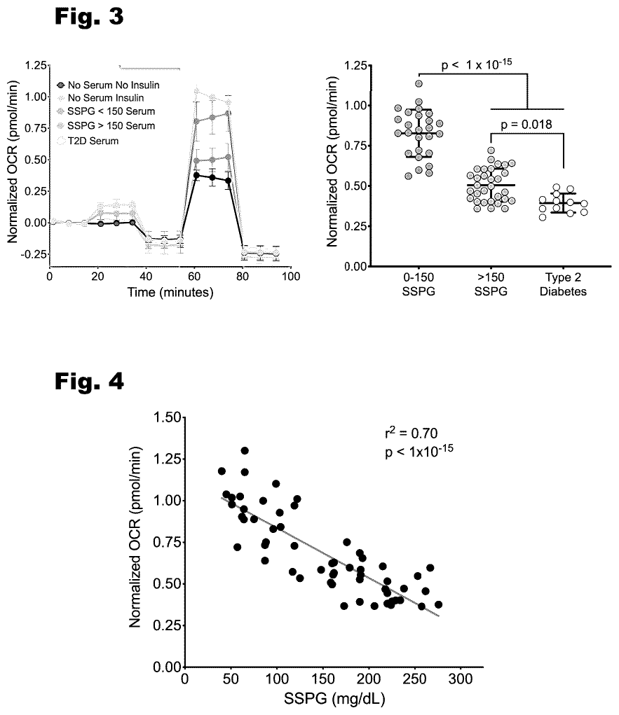

Various embodiments are directed towards assessment of insulin resistance via a mitochondrial function assay. As described in the Exemplary Embodiments, it is now known that mitochondrial response to insulin is altered by an individual's IgG, such as IgG within an individual's serum. In particular, it was found that a healthy individual's serum (or isolated IgGs from serum) had a higher maximal mitochondrial respiratory response than individuals that are insulant resistant. FIG. 3 provides an exemplary respiratory response pattern of insulin-stimulated mitochondria treated with serum of healthy individuals (i.e., individuals having SSPG≤150). insulin resistant individuals (i.e., individuals having SSPG>150), and diagnosed type 2 diabetic (T2D) individuals. Further, it was found that the insulin-stimulated mitochondrial respiratory response correlated the level of insulin resistance, as determined by an insulin suppression test (FIG. 4). These discoveries allow for a facile biological assay to determine an individual's insulin resistance based on the subject's IgG sample. Further, in many embodiments, the insulin-stimulated mitochondrial respiratory response assay is utilized as a surrogate of the insulin suppression test.

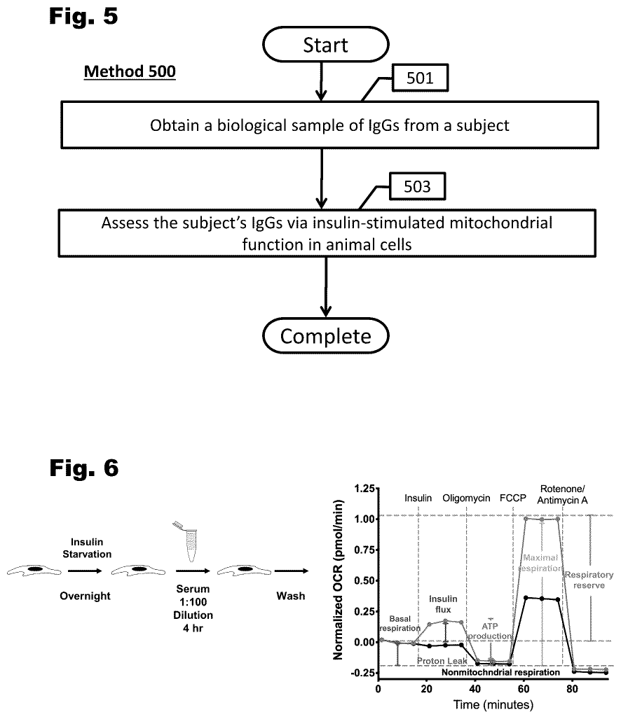

A process for assessing a subject's insulin resistance utilizing a sample of IgGs from the subject, in accordance with various embodiments, is shown in FIG. 5. This process is directed to determining an indication of insulin resistance of a subject, which can be used as diagnostic to identify subjects having an insulin resistant, hyperglycemic, and/or type 2 diabetic pathology. In some instances, the process is used a surrogate for the insulin suppression test to determine steady-state plasma glucose.

The method of FIG. 5 begins with obtaining 501 a biological sample of IgGs, the sample collected from a subject. Any appropriate biological sample containing the subject's IgGs can be utilized, including (but not limited to), blood and serum. In some instances, IgGs are enriched and/or isolated from the biological sample and used for assessment. Any appropriate subject having IgG can be utilized, including (but not limited to) humans, animal models, and animals under veterinary care.

The method of FIG. 5 also assesses 503 the subject's IgGs via an insulin-stimulated mitochondrial function in animal cells. A number of different means can be utilized to assess insulin-stimulated mitochondrial function. Generally, animal cells are insulin starved for a time period, then treated with the subject's IgG sample, and then mitochondrial respiration is measured.

An exemplary process to measure insulin resistance via mitochondrial function within animal cells in response to a subject's IgG sample is provided in FIG. 6. Any appropriate animal cell having mitochondria and expressing an IgG Fc receptor can be utilized. In some embodiments, hepatocytes or skeletal muscle cells are utilized, each of which have high mitochondrial activity and appropriate receptors.

The process can begin with starving animal cells of insulin and IgG for a period of time (e.g., overnight). In some instances, cells are kept in their respective media but lack serum or growth factor supplements. Any appropriate period of starvation can be utilized such that the cells reach a basal insulin signaling response. In some instances, animal cells are starved for a minimum of 4 hours, a minimum of 6 hours, a minimum of 8 hours, a minimum of 10 hours, or a minimum of 12 hours. Ideally, the period of starvation is long enough to reach a basal insulin signaling response in the animal cells.

After a period of starvation, a subject's IgG sample are used to treat the cells for a period of time. In some embodiments, the subject's IgG sample is simply added to media of the animal cells, but any method to treat the cells with a subject's IgG sample can be utilized. Any appropriate period of time of IgG treatment can be utilized to stimulate an IgG Fc receptor signaling response in the animal cells. In some instances, animal cells are treated for a minimum of 0.5 hours, a minimum of 1 hour, a minimum of 1.5 hours, a minimum of 2 hours, a minimum of 3 hours, or a minimum of 4 hours. In some embodiments, the IgG sample is serum or blood. In some embodiments, a subject's serum or blood is processed prior to treatment. In some embodiments, the IgG is isolated or enriched IgG.

After a period of time of IgG treatment, the animal cells are assessed for insulin-stimulated mitochondrial function. Mitochondrial function can be measured in any appropriate manner. For example, mitochondrial function can be measured by oxygen consumption rate (OCR) following insulin stimulation and pharmacological perturbations of the electron transport chain (ETC). To perform the OCR assessment, animal cells are treated with insulin to stimulate an insulin response, then treated with an ATP synthase inhibitor, then treated with a mitochondrial uncoupler to yield a maximal respiration response. Inhibitors of mitochondrial complex I and complex III can be utilized to stop mitochondrial uncoupling and end the assay.

Any appropriate insulin peptide, insulin mimic, or compound that stimulates insulin receptors can be utilized. In some embodiments, insulin is administered to the cells at a concentration between 10 nM and 10 μM. In some embodiments, insulin is administered to the cells at a concentration less than 10 nM, at a concentration between 10 nM and 100 nM, at a concentration between 100 nM and 1000 nM, at a concentration between 1000 nM and 10 μM, or at a concentration greater than 10 μM.

Any appropriate compound to perturb the ETC can be utilized, many of which are known in the art. In some embodiments, an ATP synthase inhibitor is an oligomycin and is administered at a concentration between 100 nM and 100 μM. In some embodiments, the oligomycin is administered to the cells at a concentration less than 100 nM, at a concentration between 100 nM and 1000 nM, at a concentration between 1000 nM and 10 μM, at a concentration between 10 μM and 100 μM, or at a concentration greater than 100 μM. In some embodiments, a mitochondrial uncoupler is carbonyl cyanide-4-(trifluoromethoxy)phenylhydrazone (FCCP) and is administered at a concentration between 100 nM and 100 μM. In some embodiments, FCCP is administered to the cells at a concentration less than 100 nM, at a concentration between 100 nM and 1000 nM, at a concentration between 1000 nM and 10 μM, at a concentration between 10 μM and 100 μM, or at a concentration greater than 100 μM.

Any appropriate compound to stop mitochondrial uncoupling in order to the end the assay can be utilized, many of which are known in the art. In some embodiments, a mitochondrial complex I inhibitor is rotenone and is administered at a concentration between 100 nM and 100 μM. In some embodiments, rotenone is administered to the cells at a concentration less than 100 nM, at a concentration between 100 nM and 1000 nM, at a concentration between 1000 nM and 10 μM, at a concentration between 10 μM. and 100 μM, or at a concentration greater than 100 μM. In some embodiments, a mitochondrial complex III inhibitor is antimycin A and is administered at a concentration between 100 nM and 100 μM. In some embodiments, antimycin A is administered to the cells at a concentration less than 100 nM, at a concentration between 100 nM and 1000 nM, at a concentration between 1000 nM and 10 μM, at a concentration between 10 μM and 100 μM, or at a concentration greater than 100 μM.

In several embodiments, a maximal respiration response is the oxygen consumption rate after administration between the treatment with a mitochondrial uncoupler and the treatment with mitochondrial complex I and complex III inhibitors. An example of OCR response is provided in FIG. 3, which shows responses for a cohort of healthy individuals, insulin resistant individuals, and type 2 diabetic individuals. This response shows that healthy individuals can be delineated from insulin resistant individuals. Furthermore, the maximal respiration response correlates with insulin resistance measurement steady state plasma glucose (SSPG) levels as determined by the insulin suppression test (FIG. 4). Accordingly, in some embodiments, a maximal respiration response is utilized to estimate SSPG levels of an individual, and/or to diagnose an individual as insulin resistant.

In several embodiments, determining insulin induced mitochondrial respiration after treatment with a subject's IgG sample is used to substitute other insulin resistance tests, such as (for example) the insulin suppression test. In various embodiments, determining insulin induced mitochondrial respiration after treatment with a subject's IgG sample is used as a precursor indicator to determine whether to perform a further clinical test, such as (for example) oral glucose tolerance test.

In various embodiments, the results of insulin induced mitochondrial respiration after treatment with a subject's IgG sample is utilized a diagnostic to infer insulin resistance. Based on results, if an individual is determined to be insulin resistant, the individual can be further assessed with periodic medical checkups, blood tests (e.g., HbA1c, glucose), glucose-level monitoring, and any combination thereof. In some instances, if an individual is determined to be insulin resistant, the individual can be treated to mitigate and/or prevent hyperglycemia. In some instances, a treatment is administration of a medication (e.g., metformin) and/or dietary supplement (e.g., coenzyme Q). In some instances, a treatment is an alteration to diet and/or an increase in physical activity.

While specific examples of determining a subject's insulin resistance via insulin-induced mitochondrial function are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining a subject's insulin resistance via insulin-induced mitochondrial function appropriate to the requirements of a given d can be utilized in accordance with various embodiments of the disclosure.

### Assessment of Insulin Resistance via Immunoglobulin Sialylation

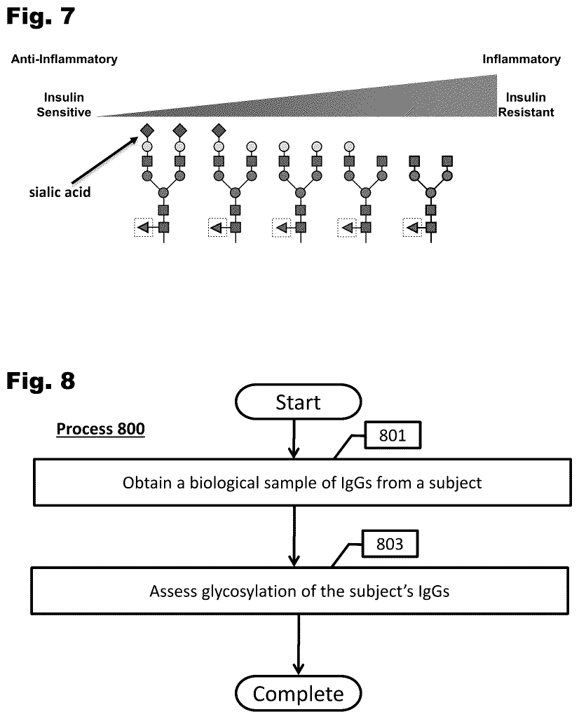

Various embodiments are directed towards assessment of insulin resistance via assessment of IgG glycosylation patterns. As described in the Exemplary Embodiments, it is now known that IgG glycosylation patterns correlate with insulin resistance (FIG. 7). In particular, it was found that IgG of healthy subjects had higher concentrations of sialylated IgG than individuals that are insulin resistant. These discoveries allow for a facile biological assay to determine a subject's insulin resistance based on the subject's sialylation of IgGs.

A process for assessing a subject's insulin resistance utilizing a sample of IgGs from the subject, in accordance with various embodiments, is shown in FIG. 8. This process is directed to determining an indication of insulin resistance of an individual, which can used as diagnostic to identify subjects having an insulin resistant, hyperglycemic, and/or type 2 diabetic pathology. In some instances, the process is used a surrogate for the insulin suppression test to determine steady-state plasma glucose.

Process 800 begins with obtaining 801 a biological sample of IgGs, which is collected from a subject. Any appropriate biological sample containing the subject's IgGs can be utilized, including (but not limited to), blood and serum. In some instances, IgGs are enriched from the biological sample and used for assessment. Any appropriate subject having IgG can be utilized, including (but not limited to) humans, animal models, and animals under veterinary care.

Process 800 also assesses 803 glycosylation (especially sialyation) of the subject's IgGs. A number of different means can be utilized to assess glycosylation and/or sialylation. Generally, the subject's IgGs are enriched and/or isolated, and then level of glycosylation and/or sialylation is measured. Glycosylation and sialylation can be measured by any appropriate methodology, including (but not limited to) neuroamidase activity, lectin binding, liquid chromatography, glycan-specific antibody binding, saccharide-specific antibody binding, sialic-acid-specific antibody binding, glycan oxidation, saccharide oxidation, and sialic acid oxidation.

In several embodiments, glycosylation and/or sialylation levels of IgG is utilized to delineate healthy individuals, insulin resistant individuals, and type 2 diabetic individuals. In some embodiments, determining a subject's glycosylation and/or sialylation levels of IgG is used to substitute other insulin resistance tests, such as (for example) the insulin suppression test. In various embodiments, determining a subject's sialylation levels of IgG is used as a precursor indicator to determine whether to perform a further clinical test, such as (for example) oral glucose tolerance test.

In various embodiments, the results of a subject's sialylation levels of IgG are utilized a diagnostic to infer insulin resistance. Based on the results, if an individual is determined to be insulin resistant, the individual can be further assessed with periodic medical checkups, blood tests (e.g., HbA1c, glucose), glucose-level monitoring, and any combination thereof. In some instances, if an individual is determined to be insulin resistant, the individual can be treated to mitigate and/or prevent hyperglycemia. In some instances, a treatment is administration of a medication (e.g., metformin) and/or dietary supplement (e.g., coenzyme Q). In some instances, a treatment is an alteration to diet and/or an increase in physical activity.

While specific examples of processes for determining a subject's insulin resistance via their IgG sialylation are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for a subject's insulin resistance via their IgG sialylation appropriate to the requirements of a given application can be utilized in accordance with various embodiments of the disclosure.

### Immunoglobins for Use as Medicaments

Various embodiments are directed to utilizing IgG for the treatment of insulin resistance and/or diabetes. In many embodiments, an individual is administered IgG to mitigate insulin resistance and/or prevent onset of hyperglycemia. As described in the Exemplary Embodiments, it is now known that administration of IgG improves insulin sensitivity. In particular, it was found that a single intraperitoneal injection of IgG (dose: 1 g/kg) provided an immediate improvement in glucose homeostasis in diabetic mice that was maintained over 35 days. Furthermore, it was found that sialylated IgG promoted improved glucose homeostasis. These discoveries provide a treatment approach for insulin resistance.

In some embodiments, IgG is utilized within a medicament to improve an individual's insulin sensitivity. In some embodiments, IgG is utilized within a medicament to improve an individual's glucose tolerance. In some embodiments, IgG is utilized to improve an individual's β cell function. In some embodiments, IgG is utilized within a medicament to reduce adipose tissue inflammation in an individual. IgG can be utilized within a medicament for individuals having insulin resistance, hyperglycemia, prediabetes, and/or type 2 diabetes.

In some embodiments, glycosylated IgG is utilized within a medicament to improve an individual's insulin sensitivity. In some embodiments, glycosylated IgG is utilized within a medicament to improve an individual's glucose tolerance. In some embodiments, glycosylated IgG is utilized within a medicament to improve an individual's β cell function. In some embodiments, glycosylated IgG is utilized within a medicament to reduce adipose tissue inflammation in an individual. Glycosylated IgG can be utilized within a medicament for individuals having insulin resistance, hyperglycemia, prediabetes, and/or type 2 diabetes.

In some embodiments, sialylated IgG is utilized within a medicament to improve an individual's insulin sensitivity. In some embodiments, sialylated IgG is utilized within a medicament to improve an individual's glucose tolerance. In some embodiments, sialylated IgG is utilized within a medicament to improve an individual's 13 cell function. In some embodiments, sialylated IgG is utilized within a medicament to reduce adipose tissue inflammation in an individual. Sialylated IgG can be utilized within a medicament for individuals having insulin resistance, hyperglycemia, prediabetes, and/or type 2 diabetes.

In some embodiments, a subject is administered a medicament comprising IgG to improve the subject's insulin sensitivity. In some embodiments, a subject is administered a medicament comprising IgG to improve the subject's glucose tolerance. In some embodiments, a subject is administered a medicament comprising IgG to improve the subject's β cell function. In some embodiments, a subject is administered a medicament comprising IgG to reduce adipose tissue inflammation in the subject. Subjects to be administered a medicament comprising IgG include (but are not limited to) subjects having insulin resistance, hyperglycemia, prediabetes, and/or type 2 diabetes.

In some embodiments, a subject is administered a medicament comprising glycosylated IgG to improve the subject's insulin sensitivity. In some embodiments, a subject is administered a medicament comprising glycosylated IgG to improve the subject's glucose tolerance. In some embodiments, a subject is administered a medicament comprising glycosylated IgG to improve the subject's β cell function. In some embodiments, a subject is administered a medicament comprising glycosylated IgG to reduce adipose tissue inflammation in the subject. Subjects to be administered a medicament comprising glycosylated IgG include (but are not limited to) subjects having insulin resistance, hyperglycemia, prediabetes, and/or type 2 diabetes.

In some embodiments, a subject is administered a medicament comprising sialylated IgG to improve the subject's insulin sensitivity. In some embodiments, a subject is administered a medicament comprising sialylated IgG to improve the subject's glucose tolerance. In some embodiments, a subject is administered a medicament comprising sialylated IgG to improve the subject's β cell function. In some embodiments, a subject is administered a medicament comprising sialylated IgG to reduce adipose tissue inflammation in the subject. Subjects to be administered a medicament comprising sialylated IgG include (but are not limited to) subjects having insulin resistance, hyperglycemia, prediabetes, and/or type 2 diabetes.

In some embodiments, an IgG Fc peptide is utilized within a medicament to improve an individual's insulin sensitivity. In some embodiments, an IgG Fc peptide is utilized within a medicament to improve an individual's glucose tolerance. In some embodiments, an IgG Fc peptide is utilized within a medicament to improve an individual's β cell function. In some embodiments, an IgG Fc peptide is utilized within a medicament to reduce adipose tissue inflammation in an individual. An IgG Fc peptide can be utilized within a medicament for individuals having insulin resistance, hyperglycemia, prediabetes, and/or type 2 diabetes.

In some embodiments, a glycosylated IgG Fc peptide is utilized within a medicament to improve an individual's insulin sensitivity. In some embodiments, a glycosylated IgG Fc peptide within a medicament is utilized within a medicament to improve an individual's glucose tolerance. In some embodiments, a glycosylated IgG Fc peptide is utilized within a medicament to improve an individual's β cell function. In some embodiments, a glycosylated IgG Fc peptide is utilized within a medicament to reduce adipose tissue inflammation in an individual. A glycosylated IgG Fc peptide can be utilized within a medicament for individuals having insulin resistance, hyperglycemia, prediabetes, and/or type 2 diabetes.

In some embodiments, a sialylated IgG Fc peptide is utilized within a medicament to improve an individual's insulin sensitivity. In some embodiments, a sialylated IgG Fc peptide is utilized within a medicament to improve an individual's glucose tolerance. In some embodiments, a sialylated IgG Fc peptide G is utilized within a medicament to improve an individual's β cell function. In some embodiments, a sialylated IgG Fc peptide is utilized within a medicament to reduce adipose tissue inflammation in an individual. A sialylated IgG Fc peptide can be utilized within a medicament for individuals having insulin resistance, hyperglycemia, prediabetes, and/or type 2 diabetes.

In some embodiments, a subject is administered a medicament comprising an IgG Fc peptide to improve the subject's insulin sensitivity. In some embodiments, a subject is administered a medicament comprising an IgG Fc peptide to improve the subject's glucose tolerance. In some embodiments, a subject is administered a medicament comprising an IgG Fc peptide to improve the subject's β cell function. In some embodiments, a subject is administered a medicament comprising an IgG Fc peptide to reduce adipose tissue inflammation in the subject. Subjects to be administered a medicament comprising an IgG Fc peptide include (but are not limited to) subjects having insulin resistance, hyperglycemia, prediabetes, and/or type 2 diabetes.

In some embodiments, a subject is administered a medicament comprising a glycosylated IgG Fc peptide to improve the subject's insulin sensitivity. In some embodiments, a subject is administered a medicament comprising a glycosylated IgG Fc peptide to improve the subject's glucose tolerance. In some embodiments, a subject is administered a medicament comprising a glycosylated IgG Fc peptide to improve the subject's β cell function. In some embodiments, a subject is administered a medicament comprising a glycosylated IgG Fc peptide to reduce adipose tissue inflammation in the subject. Subjects to be administered a medicament comprising a glycosylated IgG Fc peptide include (but are not limited to) subjects having insulin resistance, hyperglycemia, prediabetes, and/or type 2 diabetes.

In some embodiments, a subject is administered a medicament comprising a sialylated IgG Fc peptide to improve the subject's insulin sensitivity. In some embodiments, a subject is administered a medicament comprising a sialylated IgG Fc peptide to improve the subject's glucose tolerance. In some embodiments, a subject is administered a medicament comprising a sialylated IgG Fc peptide to improve the subject's β cell function. In some embodiments, a subject is administered a medicament comprising a sialylated IgG Fc peptide to reduce adipose tissue inflammation in the subject. Subjects to be administered a medicament comprising a sialylated IgG Fc peptide include (but are not limited to) subjects having insulin resistance, hyperglycemia, prediabetes, and/or type 2 diabetes.

It is to be understood that the various proteins and peptides utilized for treatment and/or administration can be truncated, modified, chimerized, and/or conjugated, as would be understood in the art. In some embodiments, a specific region of a protein or a peptide (e.g., Fc region of IgG or portion thereof) are truncated, modified, chimerized, and/or conjugated.

In some embodiments, a chimeric Ig is utilized within a medicament to improve an individual's insulin sensitivity. In some embodiments, a chimeric Ig is utilized within a medicament to improve an individual's glucose tolerance. In some embodiments, a chimeric Ig is utilized within a medicament to improve an individual's 13 cell function. In some embodiments, a chimeric Ig is utilized within a medicament to reduce adipose tissue inflammation in an individual. A chimeric Ig can be utilized within a medicament for individuals having insulin resistance, hyperglycemia, prediabetes, and/or type 2 diabetes. In some embodiments, the chimeric Ig within the medicament is glycosylated. In some embodiments, the chimeric Ig within the medicament is sialylated.

In some embodiments, a subject is administered a medicament comprising a chimeric Ig to improve the subject's insulin sensitivity. In some embodiments, a subject is administered a medicament comprising a chimeric Ig to improve the subject's glucose tolerance. In some embodiments, a subject is administered a medicament comprising a chimeric Ig to improve the subject's 13 cell function. In some embodiments, a subject is administered a medicament comprising a chimeric Ig to reduce adipose tissue inflammation in the subject. Subjects to be administered a medicament comprising a chimeric Ig include (but are not limited to) subjects having insulin resistance, hyperglycemia, prediabetes, and/or type 2 diabetes. In some embodiments, the chimeric Ig within a medicament is glycosylated. In some embodiments, the chimeric Ig within a medicament is sialylated.

In some embodiments, a chimeric Ig Fc peptide is utilized within a medicament to improve an individual's insulin sensitivity. In some embodiments, a chimeric Ig Fc peptide is utilized within a medicament to improve an individual's glucose tolerance. In some embodiments, a chimeric Ig Fc peptide is utilized within a medicament to improve an individual's 13 cell function. In some embodiments, a chimeric Ig Fc peptide is utilized within a medicament to reduce adipose tissue inflammation in an individual. A chimeric Ig Fc peptide can be utilized within a medicament for individuals having insulin resistance, hyperglycemia, prediabetes, and/or type 2 diabetes. In some embodiments, the chimeric Ig Fc peptide within a medicament is glycosylated. In some embodiments, the chimeric Ig Fc peptide within a medicament is sialylated.

In some embodiments, a subject is administered a medicament comprising a chimeric Ig Fc peptide to improve the subject's insulin sensitivity. In some embodiments, a subject is administered a medicament comprising a chimeric Ig Fc peptide to improve the subject's glucose tolerance. In some embodiments, a subject is administered a medicament comprising a chimeric Ig Fc peptide to improve the subject's β cell function. In some embodiments, a subject is administered a medicament comprising a chimeric Ig Fc peptide to reduce adipose tissue inflammation in the subject. Subjects to be administered a medicament comprising a chimeric Ig Fc peptide include (but are not limited to) subjects having insulin resistance, hyperglycemia, prediabetes, and/or type 2 diabetes. In some embodiments, the chimeric Ig Fc peptide within a medicament is glycosylated. In some embodiments, the chimeric Ig Fc peptide within a medicament is sialylated.

Chimeric Igs and chimeric Ig Fc peptides for use within medicaments include any possible chimeric combination of Igs: IgG, IgM, IgA, IgD and IgE. Further, any chimeric Ig and chimeric Ig Fc peptide can utilize any combination of subclasses, such as the subclasses of IgG: IgG1, IgG2, IgG3, and IgG4. In some embodiments, the chimeric Ig or chimeric Ig Fc peptide is an IgG-IgM chimera. In some embodiments, a complex of multiple Fc peptides fused together is utilized for treatment and/or administration.

In various embodiments a compound that mimics an IgG or an IgG Fc peptide capable of stimulating a response through an IgG Fc receptor is utilized for treatment. In some embodiments, the compound mimics glycosylated an IgG or an IgG Fc peptide. In some embodiments, the compound mimics sialylated an IgG or an IgG Fc peptide.

In various embodiments, a compound that induces higher levels of endogenous sialylated IgG in a patient is utilized as a treatment. In some embodiments, an agonist of sialyltransferase to induce higher levels of endogenous sialylated IgG is utilized as a treatment. In some embodiments, an antagonist of neuroaminidase to induce higher levels of endogenous sialylated IgG is utilized as a treatment. In some embodiments, a sialic acid precursor to induce higher levels of endogenous sialylated IgG is utilized as a treatment. Sialic acid precursors include (but are not limited to) ManNAc and Neu5Ac.

In various embodiments, a subject is administered a compound that mimics sialylated Fc region of IgG capable of stimulating a response through an IgG Fc receptor. In various embodiments, a subject is administered a compound that induces higher levels of endogenous sialylated IgG in a patient. In some embodiments, a subject is administered an agonist of sialyltransferase to induce higher levels of endogenous sialylated IgG. In some embodiments, a subject is administered an antagonist of neuroaminidase to induce higher levels of endogenous sialylated IgG. In some embodiments, a subject is administered a sialic acid precursor to induce higher levels of endogenous sialylated IgG. Sialic acid precursors include (but are not limited to) ManNAc and Neu5Ac.

In some embodiments, an individual's Igs are collected from the individual and then processed to glycosylate and/or sialylate the Igs (and/or Fc peptides thereof). Once glycosylated and/or sialylated, the Igs (and/or Fc peptides thereof) are utilized as a treatment for the individual and/or administered to the individual. Methods to glycosylate and sialylate proteins (including Igs and Fc peptides) are well known and appreciated in the art.

In some embodiments, proteins, peptides and compounds described herein are utilized in a therapeutically effective amount as part of a course of treatment. As used in this context, to “treat” means to ameliorate or prophylactically prevent at least one symptom of the disorder to be treated or to provide a beneficial physiological effect. For example, one such amelioration of a symptom could be reduction of insulin resistance and one such prophylactic could be prevention of hyperglycemia. Assessment of glycemic regulation can be performed in many ways, including (but not limited to) assessing insulin resistance as described herein and assessing glycemia by insulin suppression test, OGTT, glucose levels, and HbA1c levels. While thresholds of healthy SSPG levels can vary dependent on the insulin suppression test assessment, it is typically regarded that healthy SSPG is below one of: 100 mg/dL, 150 mg/dL, or 200 mg/dL. Likewise, healthy OGTT results is typically below one of: 100 mg/dL, 140 mg/dL or 200 mg/dL.

### Applications and Treatments Related to Glycemic Regulation

Various embodiments are directed to treatments related to glycemic regulation. As described herein, a subject may have their insulin resistance indicated by various methods. Based on a subject's insulin resistance indication, the subject can be treated with various medications, dietary supplements, dietary alterations, and physical exercise regimens.

### Medications and Supplements

Several embodiments are directed to the use of medications and/or dietary supplements to treat a subject to mitigate and/or prevent glycemic dysregulation, including (but not limited to) insulin resistance and/or hyperglycemia. In some embodiments, medications and/or dietary supplements are administered in a therapeutically effective amount as part of a course of treatment. As used in this context, to “treat” means to ameliorate or prophylactically prevent at least one symptom of the disorder to be treated or to provide a beneficial physiological effect. For example, one such amelioration of a symptom could be reduction of insulin resistance and one such prophylactic could be prevention of hyperglycemia. Assessment of glycemic regulation can be performed in many ways, including (but not limited to) assessing insulin resistance as described herein and assessing glycemia by insulin suppression test, OGTT, glucose levels, and HbA1c levels. While thresholds of healthy SSPG levels can vary dependent on the insulin suppression test assessment, it is typically regarded that healthy SSPG is below one of: 100 mg/dL, 150 mg/dL, or 200 mg/dL. Likewise, healthy OGTT results is typically below one of: 100 mg/dL, 140 mg/dL or 200 mg/dL.

A therapeutically effective amount can be an amount sufficient to prevent reduce, ameliorate or eliminate the symptoms of diseases or pathological conditions susceptible to such treatment, such as, for example, diabetes, heart disease, or other diseases that are affected by elevated glycemia. In some embodiments, a therapeutically effective amount is an amount sufficient to reduce an individual's insulin resistance and/or improve an individual's glucose tolerance. In similar embodiments, a therapeutically effective amount is an amount sufficient to reduce a subject's insulin resistance or hyperglycemia result below a certain threshold.

A number of medications are available to treat elevated glycemia, such as those used to treat type II Diabetes. Medications include (but are not limited to) insulin, alpha-glucosidase inhibitors (e.g., acarbose, miglitol, voglibose), biguanides (e.g., metformin), dopamine agonists (e.g., bromocriptine), DPP-4 inhibitors (e.g., alogliptin, linagliptin, saxagliptin, sitagliptin, vildagliptin, gemigliptin, anagliptin, teneligliptin, trelagliptin, omarigliptin, evogliptin, gosogliptin, dutogliptin, berberine), GLP-1 receptor agonists (e.g., glucagon-like peptide 1, gastric inhibitory peptide, albiglutide, dulaglutide, exenatide, liraglutide, lixisenatide, semaglutide), meglitinides (e.g., nateglinide, repaglinide), sodium glucose transporter 2 inhibitors (e.g., dapagliflozin, canagliflozin, empagliflozin, ertugliflozin, ipragliflozin, luseogliflozin, sotagliflozin, tofogliflozin), sulfonylureas (e.g., glimepiride, gliclazide, glyburide, chlorpropamide, tolazamide, tolbutamide, acetohexamide, carbutamide, metahexamide, glycyclamide, glibornuride, glipizide, gliquidone, glisoxepide, glyclopyramide), and thiazolidinediones (e.g., rosiglitazone, pioglitazone, lobeglitazone). Furthermore, as described herein, a subject can be treated for insulin resistance with sialylated IgG Fc. Accordingly, an individual may be treated, in accordance with various embodiments, by a single medication or a combination of medications described herein. Furthermore, several embodiments of treatments further incorporate heart disease medications (e.g., aspirin, cholesterol and high blood pressure medications), dietary supplements, dietary alterations, physical exercise, or a combination thereof.

Numerous dietary supplements may also help to treat elevated glycemia. Various dietary supplements, such as alpha-lipoic acid, chromium, coenzyme Q10, garlic, hydroxychalcone (cinnamon), magnesium, omega-3 fatty acids, psyllium and vitamin D have been shown to have beneficial effects on individuals having diabetes and cardiac conditions. Thus, embodiments are directed to the use of dietary supplements, included those listed herein, to be used to treat a subject based on a subject's insulin resistance result. A number of embodiments are also directed to combining dietary supplements with medications, dietary alterations, and physical exercise to reduce glycemic variability.

### Diet and Exercise

Numerous embodiments are directed to dietary alteration and exercise treatments. Altering one's lifestyle, including physical activity and diet, has been shown to improve glycemic regulation. Accordingly, in a number of embodiments, an individual is treated by altering their diet and increasing physical activity in response to an insulin resistance assessment result.

There are various diets that will help different individuals in getting better glycemic control. A number of embodiments are directed to treatments to reduce weight, which has been considered by some to be the best approach to control one's glycemia. There are many programs based on the seminal study for a low-fat diet to prevent diabetes (see Diabetes Prevention Program (DPP) Research Group. Diabetes Care. 2002 25:2165-71, the disclosure of which is herein incorporated by reference). For others, a diet low in refined carbohydrates and sugars will work better. Numerous embodiments take a more personalized approach such that one can utilize continuous glucose monitoring (CGM) results to determine which foods cause glycemic spikes for an individual and devise a diet to limit these particular foods while maintaining appropriate nutrient intake. Numerous embodiments are directed to treating an individual by substituting saturated fats with monounsaturated and unsaturated fats to help lower the risk for cardiovascular disease, which would be beneficial for many individuals struggling to control their glycemia. Also, embodiments are directed to increasing amounts of fiber in the diet, which would be highly recommended to both help with glycemic regulation and also balance serum lipid levels (cholesterol and triglycerides).

Exercise has a large impact on glycemic regulation. In several embodiments, a treatment would entail a minimum of some minutes of active exercise per week. In some embodiments, treatments would include a minimum of 150 minutes of exercise a week, however, the precise duration of exercise may be dependent on the individual to be treated and their cardiovascular health. It is further noted that cardiovascular exercise is important for the immediate glycemic control and weight training will have a long-term effect by increasing muscle mass, affecting glucose utilization during rest.

In many embodiments, a treatment to help control glucose levels is stress management, as stress increases blood glucose levels. Some proven ways to help control stress include meditation, social support, adequate sleep, journaling, and therapy.

### EXEMPLARY EMBODIMENTS

Biological data support the methods of diagnostic assessments and treatments described herein. In the attached manuscript and figures, exemplary diagnostics and treatments for insulin resistance and hyperglycemia are provided.

### Example 1: Immunoglobulin G sialylation is a Biomarker and Reversible Pathogenic Cause of Insulin Resistance

Insulin resistance (IR) is a complex phenotype that defies explanation by a single etiological mechanism but results in decreased insulin-mediated glucose uptake in insulin-responsive tissues, especially skeletal muscle and adipose. Currently, quantification of insulin sensitivity is challenging and requires invasive, time-consuming assays, which are not practical for routine clinical care. Hallmarks of IR include low-grade inflammation, aberrant cytokine and hormone secretion, deregulation of lipid and amino acid metabolism, and altered composition of the gastrointestinal microbiome. These hallmarks are often used as molecular surrogates of insulin sensitivity; however, they do not robustly quantify insulin sensitivity nor do they fully account for the underlying disease biology. Their inability to recapitulate invasive measurements of insulin sensitivity may reflect the complexity of integrating the myriad concomitantly acting factors that modulate insulin sensitivity in vivo. Indeed, the development of multivariate models that incorporate the levels of multiple proteins, metabolites, and microbes can approximate quantitative assessment of IR. However, thus far, a single blood-based marker or assay that can quantify and modulate IR has not been identified and our understanding of the etiology of insulin resistance is poorly understood.

Another hallmark of IR is mitochondrial dysfunction in insulin-responsive tissues. This phenotype is characterized by several molecular features, including reduced oxidative phosphorylation, ATP synthesis, respiration capacity, metabolic plasticity, and membrane potential as well as increased proton leak, production of reactive oxygen species, and mitophagy (C. Koliaki and M. Roden, Annu Rev Nutr. 2016; 36:337-367, the disclosure of which is incorporated herein by reference). These mechanisms of dysfunction can be inherited through the mitochondrial genome or acquired over one's lifetime, possibly due to lifestyle and environmental factors. Several studies have observed these mitochondrial phenotypes in vivo and muscle biopsies from individuals with type 2 diabetes (T2D) and obesity-linked IR (G. Cline, et al. J Clin Invest. 1994; 94:2369-2376; J. Szendroedi, et al., PLoS Med. 2007; 4:e154; and K. Petersen, et al., N Engl J Med. 200;350:664-71; the disclosures of which are each incorporated herein by reference). Mitochondrial function is highly sensitive to stress and responds dynamically to the changes in the cellular environment. Recently, measurements of mitochondrial function in primary tissue samples have emerged as a biomarker of inflammation in chronic diseases, including rheumatoid arthritis and Alzheimer's disease.

The current study investigated the ability of the immune system, including circulating cytokines and hormones, to modulate mitochondrial function. Described herein is a novel personalized surrogate measurement of insulin sensitivity using insulin-stimulated mitochondrial respiration following acute exposure to an individual's serum. This simple blood-based assay closely approximates an individual's insulin sensitivity. By analyzing the mechanism of this phenomenon, it was further demonstrated that the glycosylation state of the Fc region of IgG is a determinant of insulin sensitivity. These alterations were determined to be causative because glyco-engineered antibodies can modulate and correct IR in vivo. These results have implications for the detection and monitoring of IR and type 2 diabetes, provide insight into the pathology, and provide novel therapeutic strategies for these diseases.

### Methods

Mice. Experiments were performed in male C57BL/6 (B6), B6.BKS(D)-Lepr^{db}/J (db/db) and B10.12952(B6)-Ighm^{tm1Cgn}/J (μMT) purchased from Jackson Laboratory and maintained in a pathogen-free, temperature-controlled environment on a 12-h light and dark cycle. All mice used in comparative studies were males and age-matched between groups within individual experiments. Studies used protocols approved by the Institutional Animal Care and Use Committee of Stanford University.

Human Samples. Serum was obtained from 57 individuals with metabolic phenotyping and 12 type 2 diabetic patients. Serum samples were obtained with informed consent and the approval of the Stanford Internal Review Board for Human Subjects.

Modified Insulin Suppression Test. Insulin-mediated glucose uptake was quantified by the modified version of the Insulin Suppression Test (1ST) to estimate whole-body insulin sensitivity (see J. Yip, F. S. Facchini, and G. M. Reaven, J Clin Endocrinol Metab.1998; 83:2773-2776; F. Abbasi, et al., Diabetes Res Clin Pract. 2018; 136:108-115; the disclosures of which are each incorporated herein by reference). After an overnight fast, a continuous intravenous infusion of octreotide acetate (0.27 μg/m^{2 /}min), insulin (32 mU/m^{2 /}min), and glucose (267 mg/m²/min) was given for 180 minutes. Blood samples were collected every 30 minutes until 150 minutes into the infusion and then every 10 minutes to measure the steady-state plasma insulin (SSPI) and stead-state plasma glucose (SSPG) concentration. During the 1ST, endogenous insulin secretion is suppressed by octreotide acetate and SSPI concentrations are similar among individuals. The height of SSPG concentration thus provides a direct measure of insulin-mediated glucose uptake: the higher the SSPG concentration, the more insulin resistant is the person. Insulin-mediated glucose uptake measured by the Insulin Suppression Test highly correlate with that by the Euglycemic Hyperinsulinemic Clamp (J. W. Knowles, et al., Metabolism. 2013; 62:548-553, the disclosure of which is incorporated herein by reference).

Cell Lines. HepaRG were obtained from Biopredic International and maintained in Williams E media without L-glutamine and phenol red (Lonza) containing maintenance/metabolism supplement (ThermoFisher) and GlutaMAX (ThermoFisher). C2C12 myoblast were obtained from ATCC and maintained using Dulbecco's modified Eagle's (DMEM) media containing 10% FBS and penicillin and streptomycin. C2C12 myoblast differentiation to myotubes was previously described. Briefly, C2C12 myoblast were grown until fully confluent and differentiation was induced with DMEM containing 2% horse serum for 48 hours. Media was changed over to DMEM containing 10% FBS and 100 nM insulin and changed daily. Skeletal muscle cells were obtained from Promocell and cultured in skeletal muscle growth media (Promocell) containing fetal calf serum (0.05 mL/mL), fetuin (50 μg/mL), epidermal growth factor (10 ng/mL), basic fibroblast growth factor (1 ng/ml, insulin (10 μg/ml) and dexamethasone (0.4 μg/mL). All cell lines were cultured in a humidified incubator at 37° C. with 5% CO₂.

Immunoblotting. Protein extracts were made in RIPA buffer, quantified by BCA assay, and diluted to equal concentrations with 4×LDS sample buffer and reducing reagent (Invitrogen). Polyacrylamide gel electrophoresis was performed on NuPAGE Novex gradient gels (ThermoFisher) followed by wet transfer to PVDF membranes. Blocking was performed with 5% non-fat milk for 1 hour and primary antibodies were incubated overnight at 4° C. in 5% milk. Primary antibodies included phospho-Akt (Cell Signaling), anti-Akt (Cell Signaling), anti-mouse IgG (Jackson ImmunoResearch Laboratories). Membranes were washed in PBST and then probed with HRP-conjugated secondary antibody (Cell Signaling) at room temperature for 1 hour. Membranes were washed and developed with ECL pico (Thermo Fisher). Quantification of immunoblot was performed using Image Lab software (Bio-Rad).

Mitochondrial Insulin Stimulation Test. HepaRG hepatocytes or skeletal muscle cells were plated at 40,000 cells/well in Seahorse XF96 plate (Agilent) the day before the assay. Cells were serum starved by culturing in respective media without serum or growth factor supplements. Individual serum was diluted 1:100 in supplement-free media and cells were incubated with media containing individual serum for 4 hours. Cells were washed three times with PBS and incubated with seahorse assay media (buffer free RPMI with mM glucose, mM sodium pyruvate and mM glutamine) for 1 hour in a CO₂ free incubation at 37° C. Seahorse measurements were performed every 5 minutes with mixing. Mitochondrial function was perturbed by administration of insulin (100 nM), oligomycin (1 μM), FCCP (2 μM), and rotenone and antimycin A (1 μM). Data was processed by normalizing basal respiration to zero and the maximal respiration following FCCP administration of the positive control (no serum exposure with insulin stimulation) to 1.

IgG purification and fragmentation. IgG purified from serum was diluted (1:100) in Protein G binding buffer (Thermo) and incubated with Protein G Agarose Beads (Thermo) for 4 hours on an orbital shaker at 4° C. Beads were washed three times with Protein G binding buffer and IgG was eluted by incubated beads in Protein G elution buffer, pH 2.7 for 5 minutes, centrifuged and the supernatant was quenched with Tris-HCl buffer, pH 9.0. Purified IgG was buffer exchanged to using 15 kDa spin column to PBS. Purification was validated by immunoblotting. Fab fragments were generated by papain cleavage using Pierce F(ab′)₂ Preparation Kit (Thermo Scientific) according to manufacturer's protocol.

Peptide Synthesis. SYN746 (QRFCTGHFGGLYPCHGP; SEQ ID NO: 1) and HIV TAT conjugated FcRn C-terminal tail-dominant negative (YGRKKRRQRRRGAPWISLRGDDTGVLLPTP; SEQ ID Nos: 2 and 3) were synthesized using Fmoc solid phase peptide synthesis and purified using preparative HPLC using aC18 reverse-phase column and characterized by liquid-chromatography mass spectrometry by University of Minnesota Peptide Synthesis Core.

IgG preparation, administration, and evaluation of glucose homeostasis in vivo. IgG was purified from mouse gamma globulin (Rockland) was using Protein G chromatography (ThermoFisher). Purified mouse IgG was buffer exchanged to sterile saline solution using 30 kDa Amicon Ultra-15 centrifugal filters (Millipore). Mice were fasted for four hours and administered an intraperitoneal injection of insulin (0.85 unit/kg, Humulin R; Eli Lily) for insulin tolerance test (ITT) or fasted for six hours and administered an IP injection of glucose (2 g/kg of body weight; Sigma-Aldrich) for glucose tolerance test (GTT). Tail vein blood samples were collected at 0, 15, 30, 45, 60, and 90 minutes and plasma glucose were measured by glucometer. Fasting plasma insulin concentrations were determined by ELISA (Alpco).

Sialic acid quantification. Serum sialic acid levels were measured using the periodate-resorcinol method (G. W. Jourdian, L. Dean, and S. Roseman, J Biol Chem., 1971; 246:430-435, the disclosure of which is incorporated herein by reference). Serum samples were thawed on ice and oxidized with periodic acid (Sigma, 32 mM) for five minutes at room temperature followed by incubation on ice for 35 minutes. Resorcinol reagent was added to each sample and incubated at 100° C. for 15 minutes and then cooled to 4° C. Tert-butanol was added to each sample and absorbance at 630 nm was measured. Sialic acid concentrations were interpolated from a standard curve generated using various concentrations of N-acetylneuraminic acid (Santa Cruz) as the standard.

Neuraminidase Activity. Mouse serum neuraminidase activity was detected using Amplex Red neuraminidase (sialidase) assay kit (ThermoFisher) according to the manufacturer's protocol.

Lectin ELISA. Goat anti-human IgG F(ab′)₂-fragments (Jackson ImmunoResearch Laboratories) were diluted in ELISA coating buffer (0.1 M, pH 9.6) at 2 μg/mL and applied to 96-well MaxiSorp microtiter plates (Thermo Scientific) at 4° C. overnight. Plates were washed and blocked with 5% BSA in PBS containing 0.05% Tween 20 (PBST) overnight at 4° C. Plates were incubated with diluted sera (1:1000) at room temperature for 1 hour, washed with PBST and incubated with biotinylated SNA (Vector Laboratories). Plates were washed and incubated with streptavidin conjugated HRP (BD Biosciences), washed, and developed with Amplex Red (ThermoFisher). Absorbance was measured after 15 minutes at 560 nm.

In vitro desialylation of mouse IgG. Desialylation of mouse IgG was performed in vitro as follows 10 mg of IgG in 1 ml of 0.05 M sodium citrate buffer (pH 6.0) was incubated with 1000 U of recombinant neuraminidase cloned from Clostriduim perfringens (New England BioLabs) at 37° C. overnight. IgG was purified by Protein G affinity chromatography (ThermoFisher) and buffer exchanged to sterile saline solution using 30 kDa Amicon Ultra-15 centrifugal filters (Millipore). Desialylation was confirmed by lectin blotting for a2,6 sialic acid with biotinylated Sambucus nigra lectin (SNA) (Vector laboratories) and streptavidin-HRP (BD Biosciences).

Statistical analysis. Statistical significance between two means was determined by unpaired Student's t-test. Statistics between three or more means was measured by One-way analysis of variance (ANOVA) and multiple hypothesis corrected using Tukey multiple comparison test. In the figure legends the number of experiments performed are listed as well as the total number of samples or mice analyzed. All seahorse respiration traces are presented as means±s.e.m. while all other plots are presented as means±s.d.

### Results

In Vitro Insulin-induced mitochondrial respiration following acute sera exposure correlates with In Vivo insulin sensitivity.

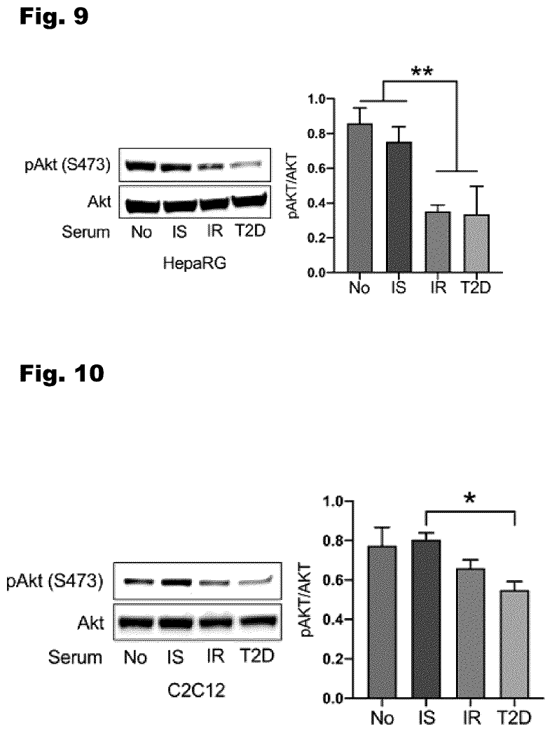

Recombinant cytokines and endogenously produced liver, muscle, and adipose tissue hormones can modulate insulin signaling in cell and mouse models. However, the direct effect of these biomolecules on insulin signaling using patient serum has not been examined. The ability of patient serum to modulate insulin signaling was investigated using two assays. First, insulin is known to activate the Akt via phosphorylation. The acute insulin signaling response of serum-starved human HepaRG hepatocytes and mouse C2C12 derived myotubes to diluted patient serum (1% final concentration) was measured. Exposure of either cell line to insulin and sub-physiological levels of serum from an IS patient or controls results in robust Akt phosphorylation. In contrast, exposure to IR or T2D patient serum resulted in a significant loss of insulin-dependent Akt phosphorylation (FIG. 9). These experiments demonstrated the ability of serum factors in IR and T2D patients to impair insulin signaling.

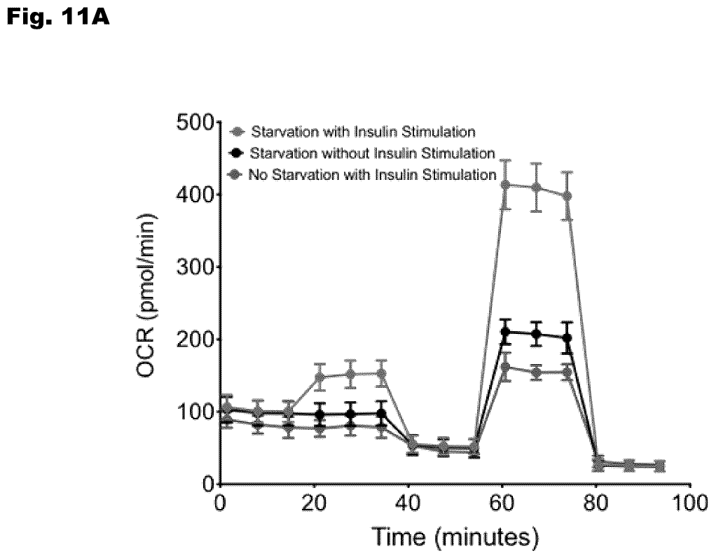

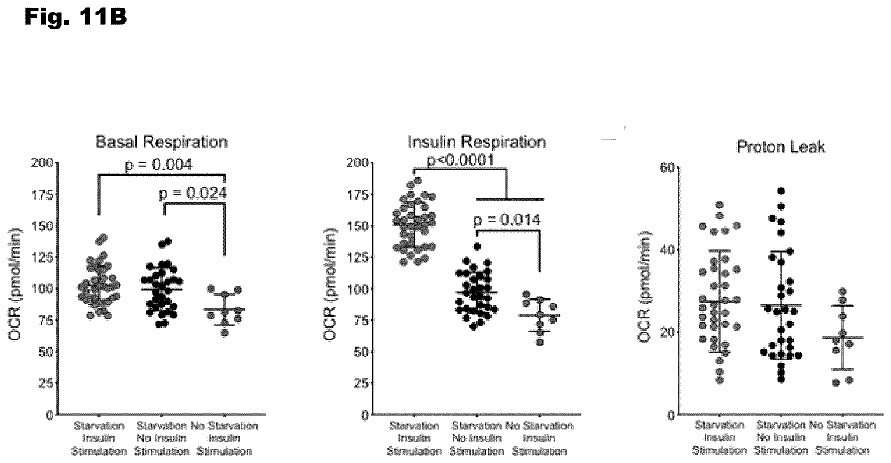

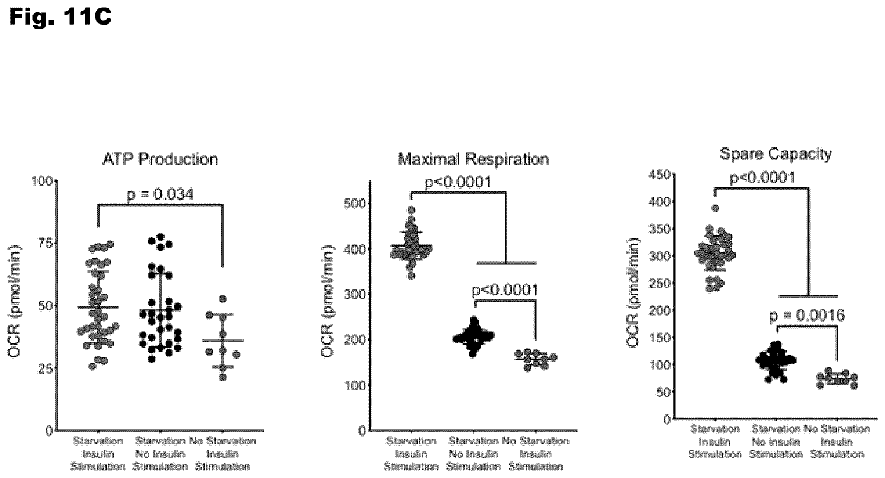

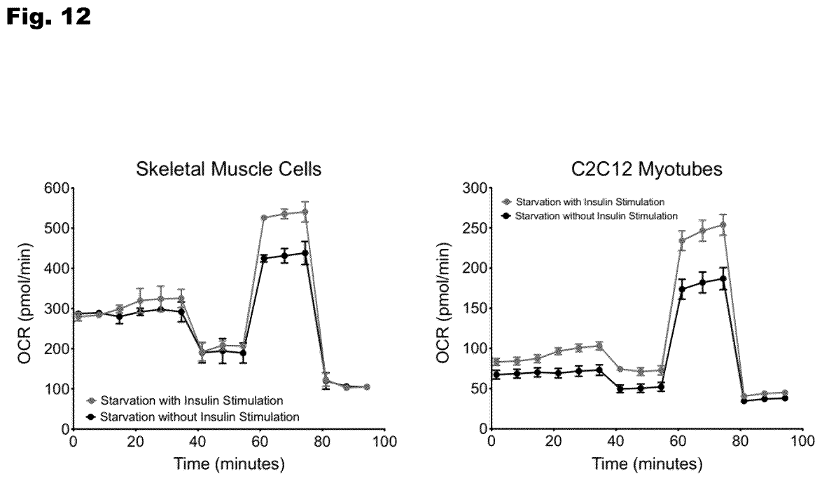

Next, a functional assay was used to investigate whether diluted patient serum altered insulin-dependent mitochondrial function, a downstream measure of insulin activity. A quantitative assessment of insulin sensitivity was developed, referred to as mitochondrial insulin stimulation test (MIST). MIST assesses mitochondrial function as measured by oxygen consumption rate (OCR) following insulin stimulation and pharmacological perturbations of the electron transport chain (ETC). Control experiments in HepaRG hepatocytes that have undergone overnight serum starvation robustly increased mitochondrial respiration and ATP production in response to insulin stimulation, resulting in a significant increase in maximal respiration and spare respiratory capacity following ETC uncoupling by carbonyl cyanide-p-trifluoromethoxyphenylhydrazone (FCCP; FIGS. 10 and 11). This effect is consistent with the observation that Akt is a stimulator of mitochondrial respiration. Hepatocytes, which were serum-starved but did not receive insulin stimulation, did not exhibit this increase indicating, that the response is insulin-dependent (FIGS. 10 and 11). In contrast, cells continuously exposed to insulin as well as glucose under conditions which simulated hyperinsulinemia and hyperglycemia observed in type 2 diabetes, exhibited impaired mitochondrial respiration to subsequent insulin stimulation and uncoupling of the ETC from ATP synthesis (FCCP; FIG. 10). Human primary skeletal muscle cells (SkMCs) and C2C12 derived myotubes also displayed insulin-dependent improvements in mitochondrial function similar to those observed for hepatocytes (FIG. 12). These results demonstrate that MIST accurately measures mitochondrial respiration in response to insulin stimulation and that diabetic levels of insulin reduces this response.

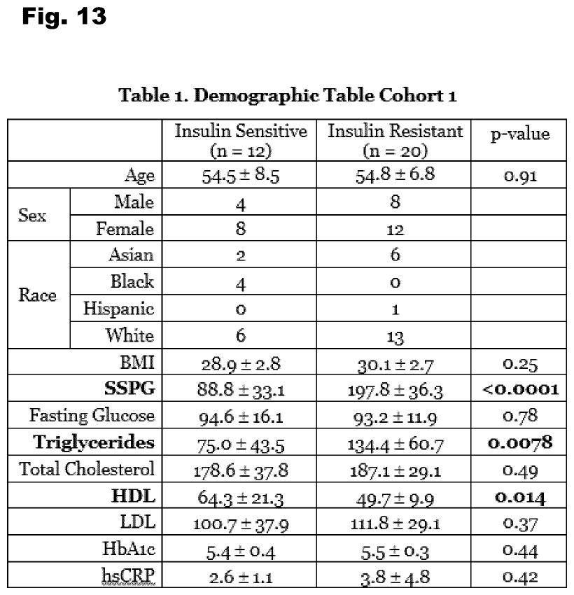

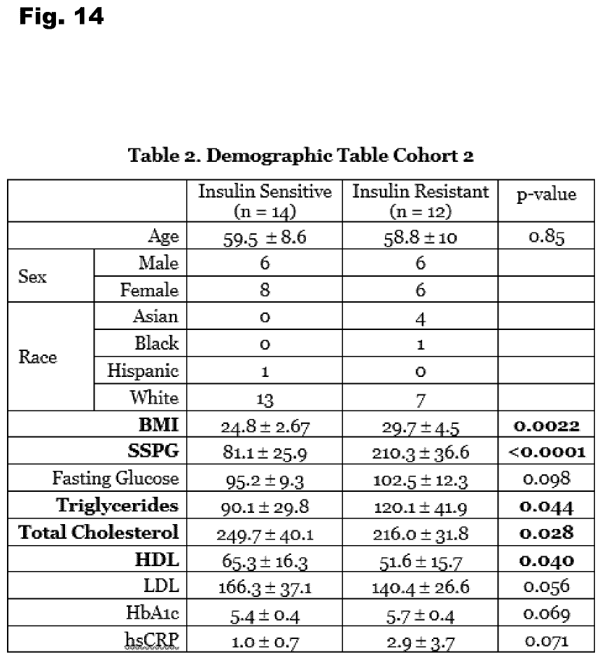

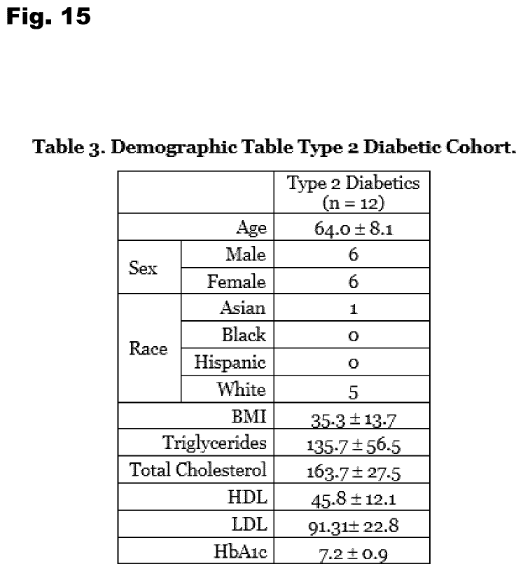

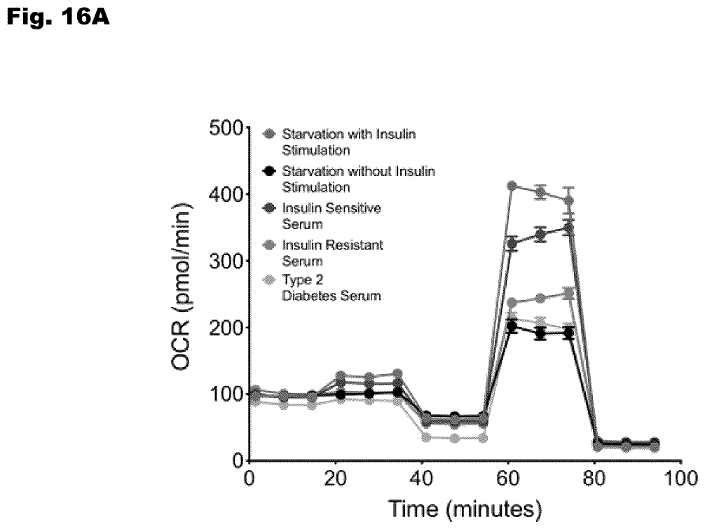

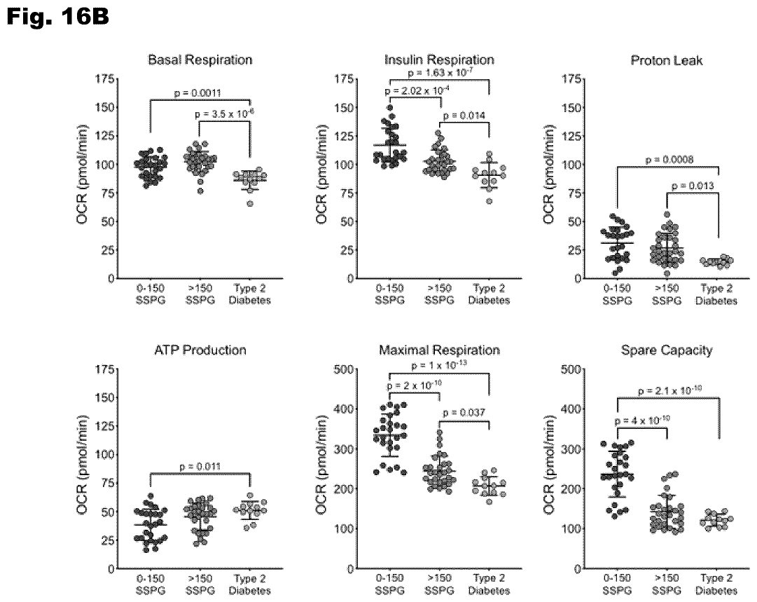

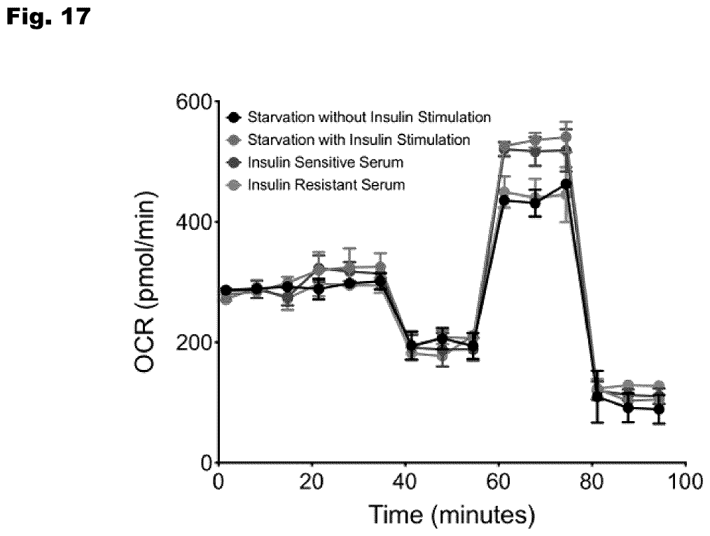

The ability of MIST to function as a surrogate measurement of insulin sensitivity was investigated by applying the assay to two independent cohorts of individuals with matched metabolic phenotyping, as well as a cohort of T2D patients (FIGS. 13-15). Individuals were metabolically phenotyped by quantifying insulin-mediated glucose disposal using the modified insulin suppression test (IST). This IST utilizes a controlled intravenous infusion of insulin and glucose to achieve a steady-state insulin concentration while glucose levels are allowed to vary (J. W. Knowles, et al., Metabolism. 2013; 62:548-553, the disclosure of which is incorporated herein by reference). The resultant steady-state plasma glucose (SSPG) level reflects the relative ability of insulin-dependent glucose disposal or peripheral insulin sensitivity. The cohorts were stratified by SSPG values, where individuals with SSPG>150 mg/dL, the 50th percentile of population-based studies using IST, were categorized as insulin resistant (IR), and those with SSPG<150 mg/dL as insulin sensitive (IS) (H. Yeni-Komshian, et al., Diabetes Care 2000; 23:171-175, the disclosure of which is incorporated herein by reference). MIST was then used to examine the cellular mitochondrial function in response to insulin following exposure to patient sera. Acute exposure of hepatocytes to sera from insulin-sensitive and resistance individuals (n=26 and 31, respectively) did not alter basal mitochondrial respiration; however, sera from T2D individuals (n=12) significantly reduced basal mitochondrial respiration (FIG. 16). Following insulin stimulation, exposure of HepaRG hepatocytes to IR and T2D sera significantly reduced insulin-induced mitochondrial respiration (p=1×10⁻⁸ and p=0.0004 respectively), maximal respiration (p<1×10¹⁵), and spare capacity compared to IS serum, indicating that IR and T2D serum reduced mitochondrial function (FIGS. 3 and 6). Human SkMCs and mouse C2C12 derived myotubes also displayed suppressed mitochondrial respiration in response to IR sera compared to IS sera (FIG. 17). These results suggest that patient serum can modulate insulin sensitivity in the MIST assay and that IR or T2D serum was sufficient to impair insulin-dependent mitochondrial function.

Maximal mitochondrial respiration observed by FCCP uncoupling of the electron transport chain is dependent on the ability of the mitochondria to build an electrochemical gradient on the outer membrane following oligomycin treatment. To examine the effect of sera on the mitochondrial membrane potential tetramethylrhodamine, methyl ester (TMRM) fluorescence was measured following serum exposure and insulin stimulation in both HepaRG hepatocytes and C2C12 myotubes. Consistent with the observed mitochondrial dysfunction measured by oxygen consumption rate, IR serum diminished mitochondrial membrane potential in response to insulin stimulation compared to IR serum. This further indicates that IR and T2D serum caused a defect in mitochondrial function.

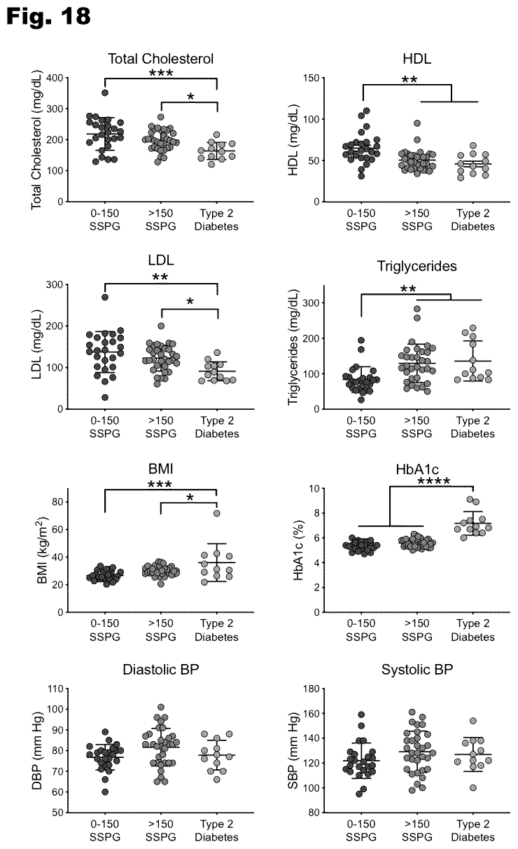

Most surprisingly, there was a strong correlation between insulin sensitivity quantified by IST and the overall insulin-dependent mitochondrial respiration in HepaRG hepatocytes following serum exposure (FIG. 4, R²=0.70, p<1×10⁻¹⁵). This correlation was not explained by differences in plasma total cholesterol, high density lipoprotein cholesterol (HDL-C), low density lipoprotein cholesterol (LDL-C), or triglycerides concentration between cohorts (FIG. 18). Additionally, the correlation between mitochondrial respiration and insulin sensitivity was significantly more robust than those observed with known, modest surrogates of IR, including fasting plasma glucose, HDL -C, or body mass index (BMI) (R²=0.0026, 0.26, and 0.15, respectively, FIG. 18). The effect of serum on mitochondrial function was also independent of other clinically measured variables linked to IR, including HbA1c and triglycerides as well (FIG. 18). Together these results demonstrate that patient-derived serum potently inhibits insulin-mediated mitochondrial function in our MIST assay and that MIST-derived values tightly correlate with in vivo measurement of insulin sensitivity.

IgG Fc region is necessary and sufficient for inhibition of insulin-dependent mitochondrial respiration.

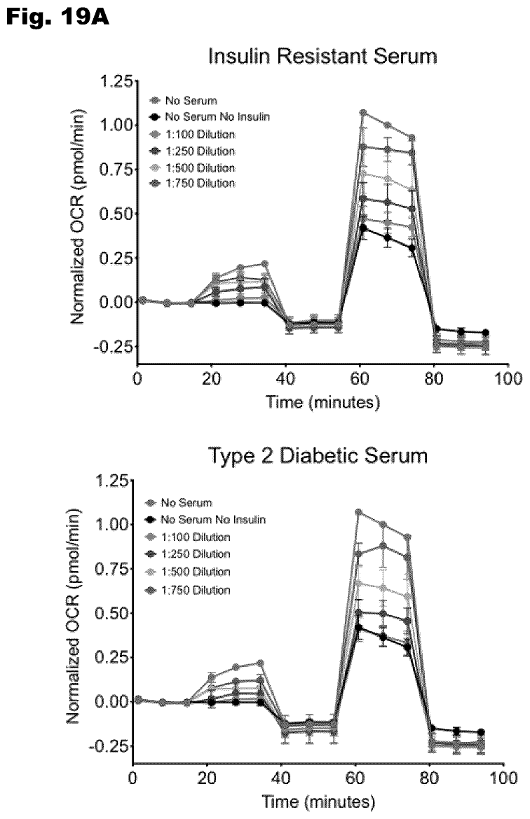

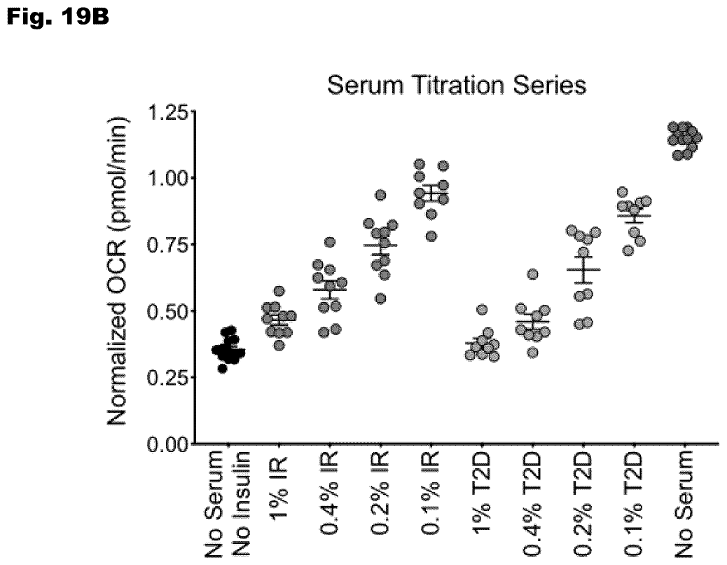

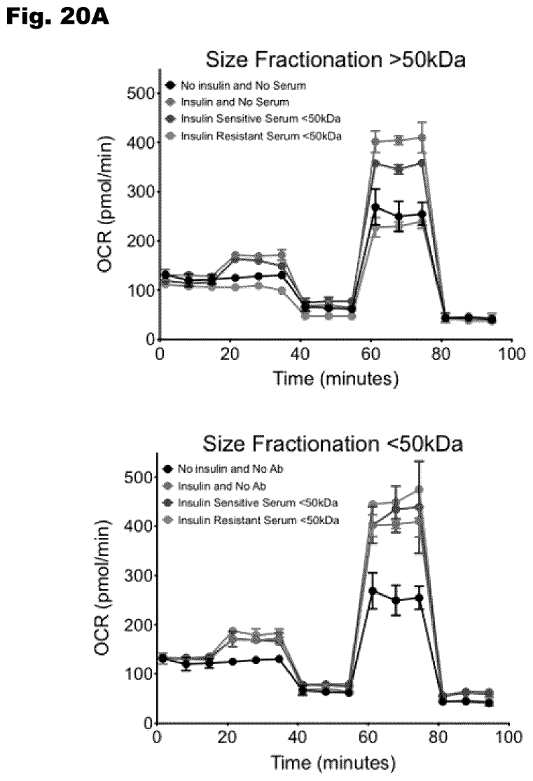

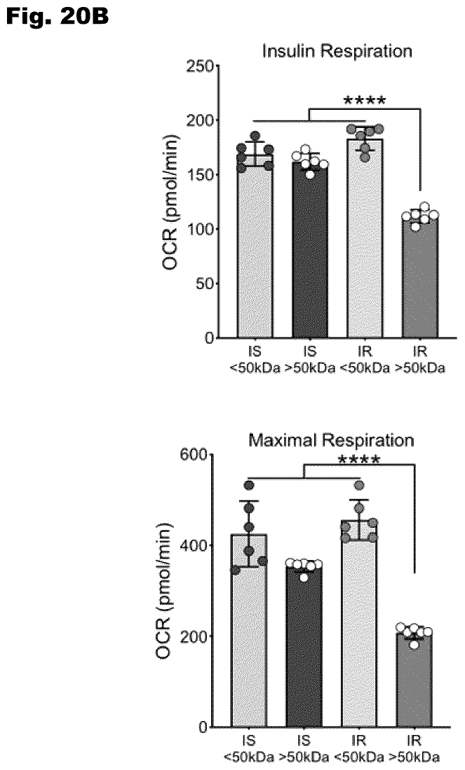

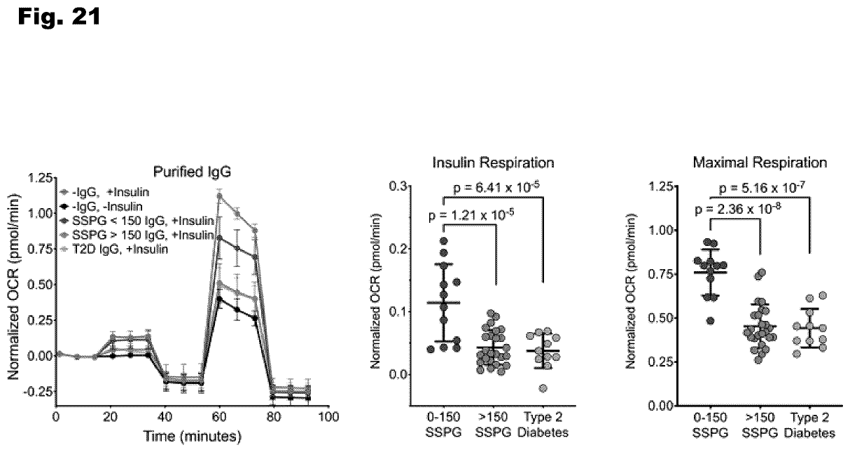

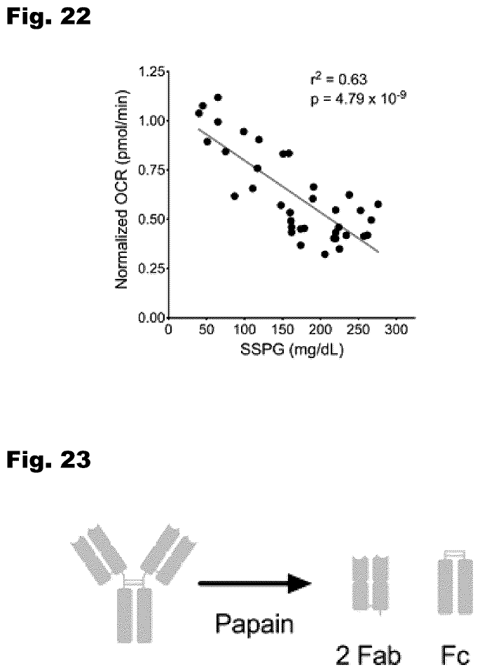

It was next sought to identify the serum factor(s) that mediate suppression of insulin-dependent mitochondrial function. Serial dilution of IR and T2D patient serum (from 1% to 0.013%) significantly inhibited mitochondrial respiration in response to acute insulin-stimulation and electron transport chain uncoupling (FIG. 19). Additionally, size fractionation of serum demonstrated that the factor(s) responsible were greater than 50 kDa (FIG. 20). These results suggested that the factor(s) were large, highly abundant protein(s). Previous reports demonstrated that IgG is capable of modulating insulin sensitivity through autoreactivity in adipose tissue as well as disrupting endothelial nitric oxide synthase (eNOS) signaling and insulin transcytosis through FcγR2B (D. A. Winer, et al., Nat Med. 2011; 17:610-617; and K. Tanigaki, et al., J Clin Invest. 2017; 128:309-322; the disclosures of which are each incorporated herein by reference). To test the hypothesis that IgG modulated insulin sensitivity and insulin-dependent mitochondrial respiration in our system, IgG was purified from patient serum and applied to the mitochondrial insulin stimulation test. Similar to treatment with diluted serum, exposure of HepaRG hepatocytes to purified IgG from IR and T2D patients significantly impaired the mitochondrial respiratory response to insulin (p=1.21×10⁻⁵ and 6.41×10⁻⁵ respectively) as well as electron transport chain uncoupling (p=2.36×10⁻⁸ and 5.16×10⁻⁷ respectively) as compared to serum from IS individuals (FIG. 21). Additionally, patient insulin sensitivity, as determined by the modified IST, correlated with mitochondrial respiration elicited by exposure to purified IgG, in agreement with the measurements made using total serum (FIG. 22, R²=0.63, p=4.79×10⁻⁹). These results indicate that IgG is the primary circulating factor mediating insulin sensitivity.

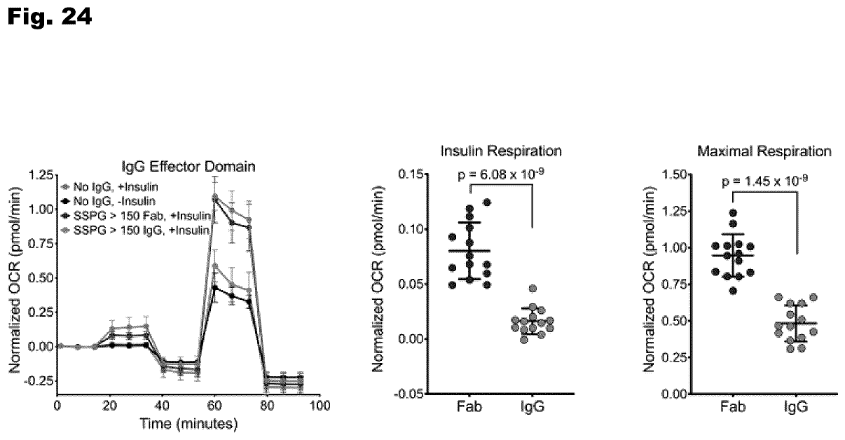

Previous studies examining the mechanism of IgG in IR examined both autoantigen recognition through the Fab domain and effector cell modulation through the Fc region (D. A. Winer, et al., Nat Med. 2011; 17:610-617; and K. Tanigaki, et al., J Clin Invest. 2017; 128:309-322; the disclosures of which are each incorporated herein by reference). Purified IgG from insulin-resistant sera was fragmented by papain cleavage and Fab fragments were collected and applied to MIST to determine the domain of IgG that mediated IR (FIG. 23). Hepatocytes treated with purified Fab fragments from insulin resistant individuals did not affect the oxygen consumption rates in response to insulin stimulation or electron transport chain uncoupling compared to the control cells. In contrast, the unfragmented IgG from IR serum again showed suppression of respiration as compared to control (FIG. 24, p=6.08×10⁻⁹). These results indicate the suppression of insulin-dependent mitochondrial respiration requires the Fc region.

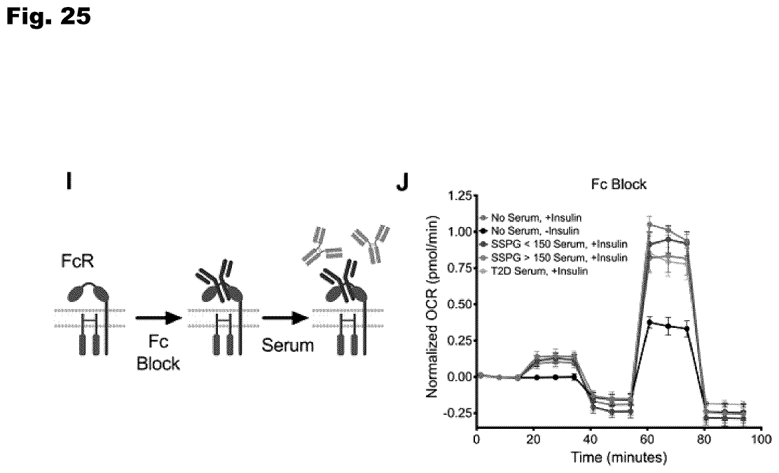

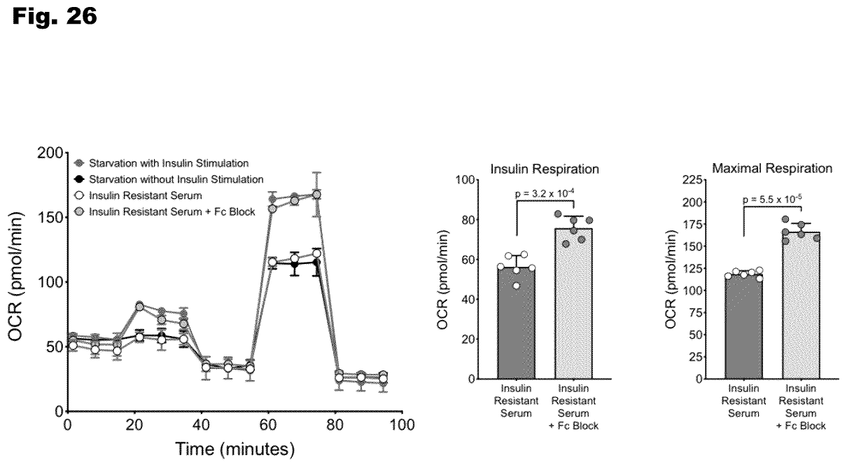

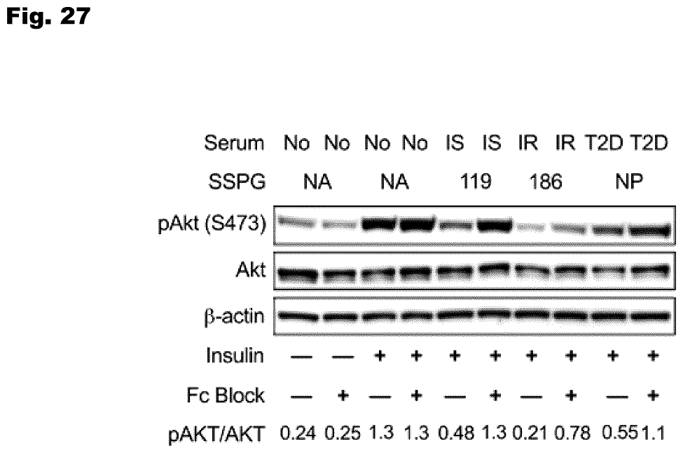

Cellular sensing of the Fc region of IgG can occur through class I or class II Fc receptors. To test if the Fc receptors were mediating the effects of IgG on mitochondrial respiration, HepaRG hepatocytes were pretreated with Fc Block reagent before exposure to patient serum MIST assay performance (FIG. 25). Remarkably, blockade of Fc receptors restored insulin sensitivity and mitochondrial respiration to cells exposed to IR and T2D sera (FIG. 25). Fc block also rescued mitochondrial respiration in human SkMCs (FIG. 26). Consistent with the recovery of insulin-dependent mitochondrial function, insulin-dependent phosphorylation of Akt was also restored following the blockade of Fc receptors (FIG. 27), although Akt phosphorylation was not as strongly correlated with measures of IR compared to MIST. Together these results demonstrated the ability of the IgG Fc region, through Fc receptor interactions, to suppress insulin-dependent mitochondrial function and insulin signaling.

FcRn is necessary for IgG suppression of insulin-mediated mitochondrial respiration.

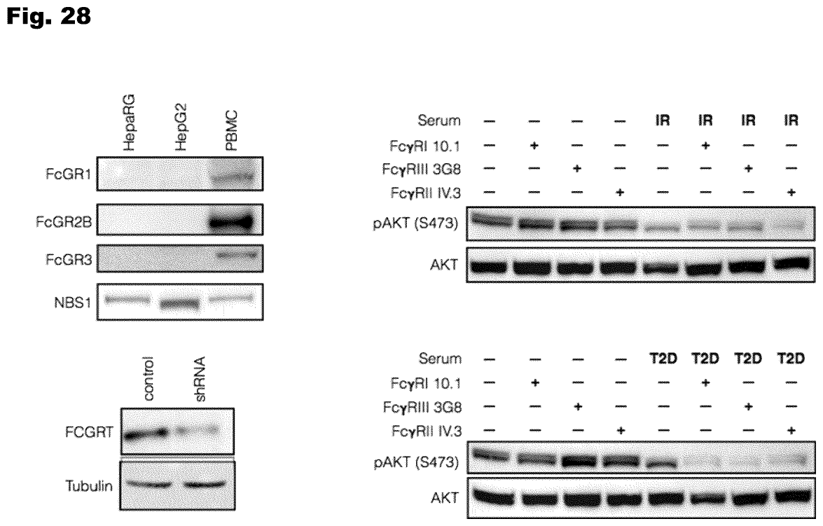

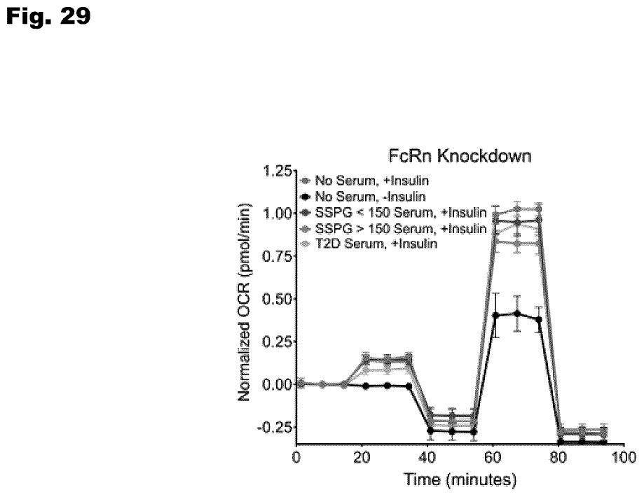

It was next sought to identify the Fc receptor responsible for the IgG-mediated suppression of insulin signaling and mitochondrial respiration. Fc receptor expression was profiled by immunoblot in HepaRG hepatocytes. This identified FcRn as the most abundant Fc receptor, whereas the classic Fc receptors (FcγRI/II/III) were not expressed (FIG. 28). Additionally, pretreatment with Fc receptor-specific functionally blocking antibodies targeting FcγRI, FcγRIIA, and FcγRIII did not rescue serum mediated IR (FIG. 28). To directly examine the ability of FcRn to mediate suppression of mitochondrial function, shRNA was used to knockdown the expression of FcRn in HepaRG hepatocytes, and then we applied diluted patient sera to the FcRn knockdown cells and measured insulin-dependent mitochondrial function using MIST. Knockdown of FcRn rescued insulin-dependent mitochondrial respiration and maximum respiration in response to ETC uncoupling, demonstrating FcRn was necessary for serum mediated disruption of insulin-dependent mitochondrial function (FIG. 29).

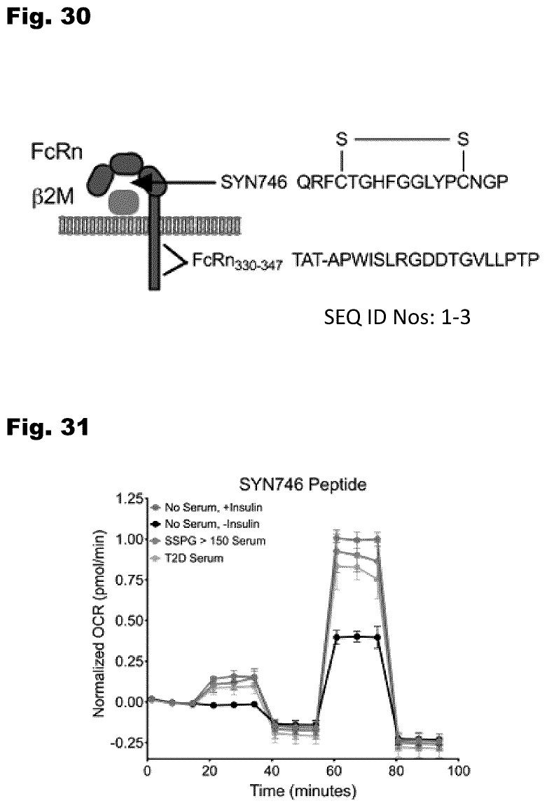

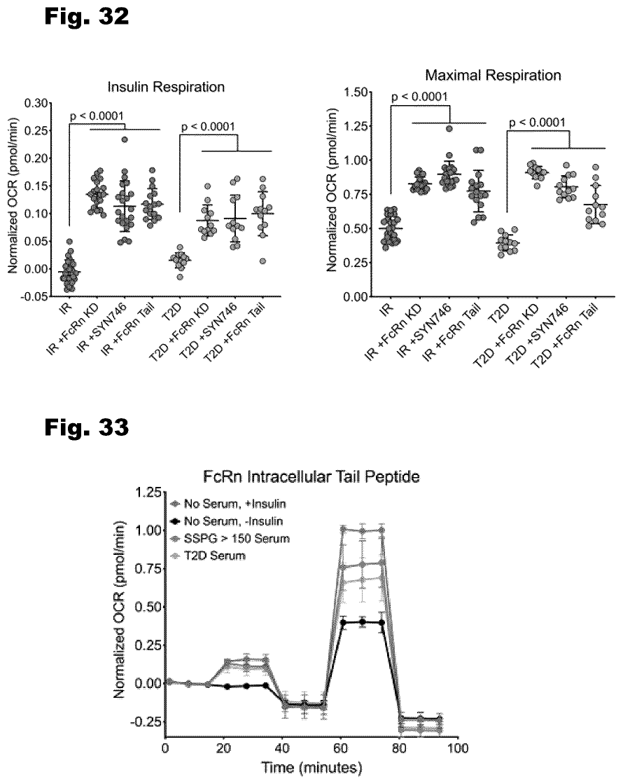

Further validation of FcRn as the receptor mediating IgG-dependent IR was investigated using two peptide-based inhibition strategies, one targeting the pH-sensitive ectodomain binding of FcRn and the other disrupting the cytoplasmic function (FIG. 30). Inhibition of IgG binding with FcRn was targeted using a previously published peptide, SYN746, which displayed pH-sensitive binding to FcRn similar to IgG. HepaRG hepatocytes were pretreated with SYN746 for 10 minutes before exposure of patient serum. SYN746 did not affect mitochondrial function or insulin stimulation in control cells. However, it provided a dramatic rescue in insulin-dependent mitochondrial respiration and maximal respiration in cells exposed to IR and T2D serum as compared to controls, which did not receive SYN746 (FIGS. 31 and 32).

Next, it was examined whether FcRn was actively involved in signaling through its cytoplasmic tail leading to reduced mitochondrial respiration following patient sera exposure. HepaRG hepatocytes were treated with a peptide consisting of the HIV TAT derived sequence (YGRKKRRQRRR; SEQ ID NO: 2) for intercellular delivery fused to the previously recognized internalization and transport motifs within the FcRn intracellular tail (APWISLRGDDTGVLLPTP; SEQ ID NO: 3) consisting of the residues 330-347 to function as a dominant-negative inhibitor of FcRn intercellular signaling. Cells were pretreated with the peptide for 10 minutes prior to exposure of serum. Control TAT peptides did not affect mitochondrial function, insulin stimulation, nor insulin sensitivity. Disruption of FcRn cytoplasmic signaling by pretreatment with the dominant negative provided a significant rescue for both insulin-dependent mitochondrial function and maximal respiration in cells treated with IR or T2D patient serum (FIGS. 32 and 33). Thus, three orthogonal approaches (genetic knockdown and two peptide-based approaches) indicated that FcRn-mediates the IgG-dependent modulation of insulin response in this system.

Administration of IgG improves insulin sensitivity and blood glucose in diabetic mice.

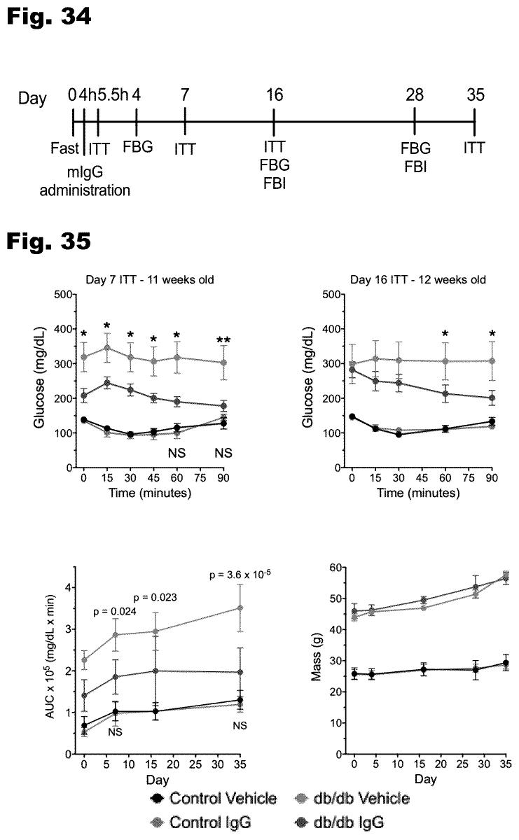

As the data indicated that IgG and FcRn can modulate IR in vitro, it was tested whether exogenous IgG administration could rescue IR in a mouse model of diabetes. Mice lacking the leptin receptor (Lepr^{db/db}, B6.BKS(D)-Lepr^{db}/J) display a complex IR-associated diabetic phenotype ²⁴. Initially, mice develop hyperglycemia that is mitigated by increased β-cell function (insulin production); however, as animals age β-cell compensation fails, and hyperglycemia gives way to an overt diabetic phenotype- a pattern that mirrors human T2D. To examine the longitudinal effects of exogenous IgG administration on insulin tolerance and glycemic regulation in vivo, intraperitoneal IgG (IPIG; dose 1 g⁻¹) was administered to hyperglycemic 10-week old Lepr^{db/db} and control C57BL6 mice, and glucose metabolism was monitored over 35 days (FIG. 34). Saline injections were used as a control. IPIG treatment did not affect body weight in either mouse model over the course of the experiment (FIG. 35).

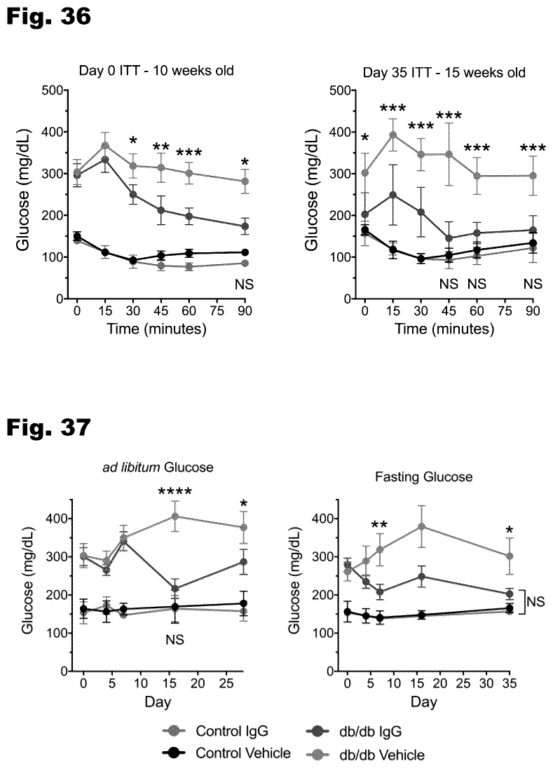

IPIG led to an immediate improvement in glucose homeostasis. At 1.5 hours post-IPIG, significantly improved insulin tolerance was detected in Lepr^{db/db} mice compared to the saline-treated control, resulting in a 21% increase in insulin-dependent glucose reduction (FIG. 4B, Cohen's d=0.72). At the same time, IPIG did not significantly affect insulin tolerance in the insulin-sensitive control mice (FIG. 4B). The improvement in insulin tolerance was maintained over the course of the experiment and enhanced with time. Starting on day 16, diabetic mice treated with IPIG achieve the same relative magnitude of response compared to control mice and achieved similar reductions in glucose levels compared to saline-treated control on Day 35 (FIGS. 35 and 36, Cohen's d =1.11). This improvement in insulin tolerance resulted in a 44% increase in insulin-dependent glucose reduction on Day 35 as compared to saline-treated Lepr^{db/db} mice. Consistent with these results, Lepr^{db/db} mice treated with IPIG had significantly decreased ad libitum glucose levels compared to untreated Lepr^{db/db} mice starting 15 days post-IPIG (FIG. 37), whereas improvements in fasting blood glucose were evident by four days post-IPIG (FIG. 37). The improvements in ad libitum glucose and FBG were maintained throughout the 35-day study period (FIG. 37). These results indicate that a single administration of IPIG improved metrics of insulin sensitivity in vivo.

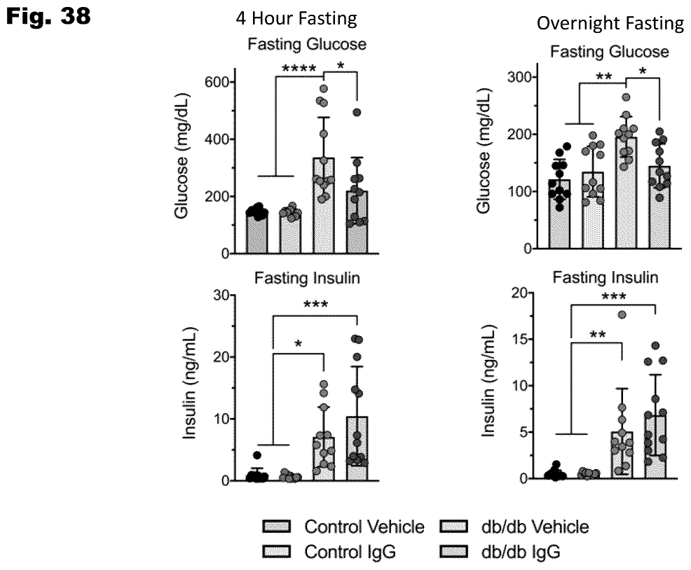

To determine the physiologic response to IPIG, static testing was used to measure fasting glucose and insulin levels. Both Lepr^{db/db} mice treated with IPIG and saline had significantly higher fasting insulin levels than control mice (FIG. 37). Similarly, twenty-eight days post-IgG administration, fasting blood glucose levels were significantly lower in IPIG treated Lepr^{db/db} mice than the vehicle treated Lepr^{db/db} mice. Fasting glucose levels were not significantly different from the those of the control mice (FIG. 38). The significant reduction in fasting glucose levels with IPIG, though still within the hyperglycemic range, did not affect fasting insulin levels in Lepr^{db/db} mice. They remained significantly elevated compared to control mice and similar to those of vehicle treated Lepr^{db/db} mice (FIG. 38). These results suggest that IPIG significantly improves glucose homeostasis in this severe diabetes model by enhanced insulin sensitivity in dynamic testing (ITT) and improved β-cell function (sustained) insulin secretion, which could result from the former or a direct response to IgG signaling.

### Conclusions

IR is a significant predictor of type 2 diabetes risk. The determination of IR has remained challenging due to the complex, laborious, and invasive nature of reference assays. Numerous surrogate measurements based on insulin and/or glucose levels exist to estimate insulin sensitivity; however, these alternatives have moderate correlations at best with gold standard assays. Here a simple cell-based in vitro assay, the mitochondrial insulin stimulation test (MIST), is capable of modeling the physiological changes in insulin sensitivity observed in vivo. This assay measures mitochondrial respiration in response to insulin stimulation. Alterations in mitochondrial function are thought to be ‘the canary in the coal mine’ for early detection of many diseases. However, measuring mitochondrial function is limited due to the need for in vivo measurements or primary tissue samples. Here, it was demonstrated that acute exposure of cells to individuals' serum modulates insulin-stimulated mitochondrial function, which is strongly correlated with insulin sensitivity (R²=0.70) as determined by the gold-standard IST. This finding establishes the ability of MIST to be a functional surrogate measurement of insulin sensitivity and suggests that serum is capable of conferring IR and mitochondrial dysfunction.

The immune system's contribution to the pathogenesis of IR through cytokine milieu is understood to regulate glucose metabolism in vivo³²⁻³⁴. Previously, the involvement of B cells in obesity-induced IR was demonstrated in studies showing the continuation of normal insulin sensitivity in B cell null mice despite the development of obesity in a DIO model. Although the B cell-mediated mechanisms regulating glucose metabolism have not been fully elucidated, B cells have been shown to participate in the deregulation of glucose metabolism through several mechanisms, including the altered cytokine production, antigen presentation, and the production of pathogenic antibodies. IgG pathogenicity has been attributed to autoreactivity and impaired insulin signaling through FcyR2B activation by the Fc region. Herein, however, IgG was identified as the serum component sufficient to mediate IR of mitochondrial function. Additionally, the results indicate that the Fc region of IgG, but not the antigen recognizing Fab, is necessary for this phenotype indicating an entirely new mechanism for insulin resistance. This result is consistent with the observation that Fab fragment transfer from DIO mice to B-cell null mice was not sufficient to cause IR in vivo.

Type I Fc receptors (FcγRI-IV) were absent in hepatocytes, but the liver and hepatocytes are a major site of the ubiquitously expressed Fc receptor, FcRn (S. Latvala, et al., J Histochem Cytochem. 2017; 65:321-333, the disclosures of which are herein incorporated by reference). Multiple lines of evidence support the role of FcRn in insulin resistance. General Fc blockade was sufficient to rescue insulin signaling, but specific inhibition of type I Fc receptors, FcγRI, IIA, and III did not. These results suggest type I Fc receptors may not participate in mediating IgG signaling events leading to IR in hepatocytes. However, in other tissues and cell types, type I Fc receptors may play a significant role, as demonstrated in endothelial cells. Furthermore, genetic knockdown of FcRn provided a robust rescue of insulin-dependent mitochondrial function upon serum exposure. Finally, two biochemical approaches, one inhibiting ectodomain binding to IgG and a dominant-negative of the intercellular tail of FcRn, both provided a robust rescue of insulin-dependent mitochondrial function. While FcRn is primarily responsible for homeostasis of circulating IgG and albumin levels, it possesses other equally critical functions, including phagocytosis and antigen presentation in podocytes, macrophages, and dendritic cells. A recent study demonstrated that hepatocyte-specific FcRn knockout did not affect circulating IgG levels, suggesting an alternative role for FcRn mediated interactions with IgG in the hepatocytes. Given the lack of type I Fc receptors in hepatocytes and their ability for antigen presentation, it is possible FcRn functions as an immune receptor in hepatocytes. Finally, FcyR deficiency in DIO did not protect mice from insulin or glucose intolerance. However, FcRn is expressed in this model and given its pervasive expression, the results offer a possible explanation for the lack of protection.

Given the involvement of IgG and FcRn in the pathogenesis of IR, the use of exogenous IgG as a therapeutic was examined. It was demonstrated that the administration of exogenous IgG provided an immediate improvement in insulin tolerance, which was not only sustained longitudinally over 35 days following a single treatment but enhanced over time. The improvement in insulin tolerance was concurrent with decreases in fed and fasting glucose levels relative to control treated diabetic mice. Together these results suggest increased insulin sensitivity, consistent with the hepatocyte in vitro results presented here. Although significant reductions in the ad libitum blood glucose levels was observed, they remained in the hyperglycemic range resulting from the polyphagia phenotype of the Lepr^{db/db} model. Accordingly, insulin levels remained appropriately elevated in both the treated and untreated diabetic mice. Nevertheless, dynamic testing of the insulin tolerance demonstrated a significant improvement in insulin sensitivity despite the hyperinsulinemia. The findings of improved glucose homeostasis in vivo suggest that modulation of IgG levels or effector functions may have promising therapeutic potential in treating IR or diabetes.

Many of the effects of IgG in vivo are attributed to Fc sialylation, but the effect of IgG Fc glycosylation on glucose metabolism has remained mostly unexplored. We identify a progressive loss of IgG sialylation with increasing obesity and IR in human sera. This study elucidates a novel link between the immune system and the regulation of glucose metabolism that may facilitate the detection and treatment of IR.

### Example 2: Treatment with IgG Reverses Diabetic Phenotypes

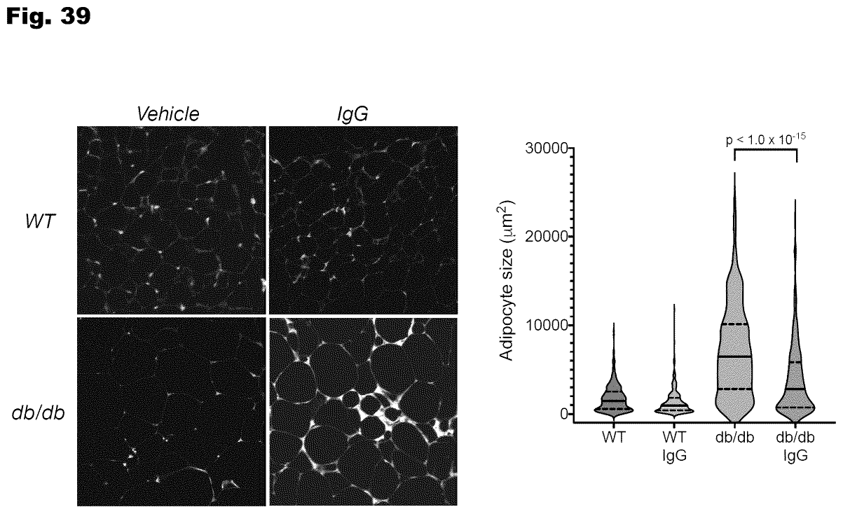

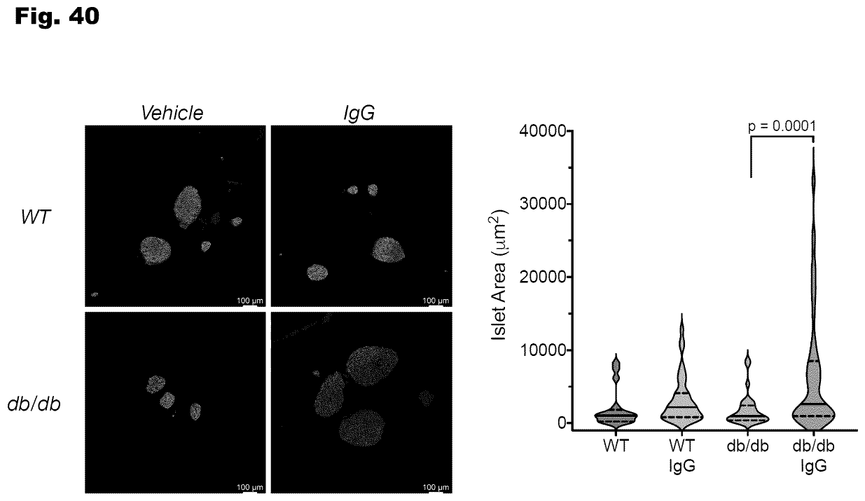

10-week old Lepr^{db/db} and wild-type mice were administered a single intraperitoneal injection of IgG (IPIG; dose 1 g kg⁻¹). Saline injections were used as a control. Mice were sacrificed 28 days after injection and tissues were harvested, fixed, sectioned, and stained. The effect of IgG on various diabetic phenotypes were examined. Diabetic mice have enlarged adipocytes compared to control, mimicking a phenotype of individuals with type 2 diabetes (FIG. 39). Treatment with IgG reverses the enlarged adipocytes of diabetic mice to a size nearing the size of adipocytes in healthy control mice (FIG. 39). Further, diabetic mice have decreased number of β cells resulting in decreased islet size (FIG. 40). Treatment with IgG reverses the decrease of β cells and islet size of diabetic mice to a size more similar to healthy control mice.

### Example 3: Multimeric IgG Fc Peptides Reduce Dosage Amount for Inducing Insulin Sensitivity

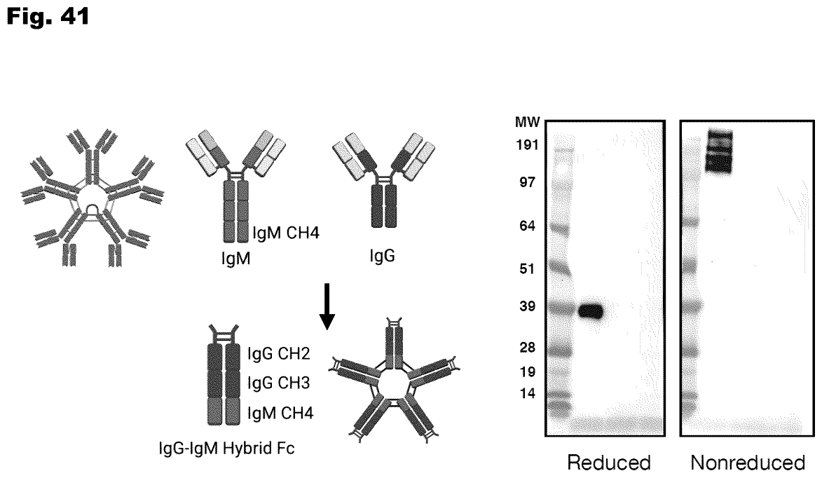

Multimeric IgG Fc peptides were generated by generating an IgG-IgM chimeric Fc peptide. Specifically, multimeric Fc peptides were generated with IgG CH2 and CH3 regions and IgM CH4 region (FIG. 41 The chimeric IgG-IgM peptide was cloned into the lentiviral vector pLenti6.3 containing a CMV promoter and IL-2 signaling sequence. Lentiviral infection was used to establish a stable expression cell line in HEK293T cells. IgG-IgM peptides were secreted from the cells into the media and purified using fast-protein liquid chromatography (FPLC) first by affinity purification using protein A and followed by size-exclusion chromatography. The generated multimeric IgG Fc peptides were analyzed via reduced and non-reduced gel electrolysis (FIG. 41).

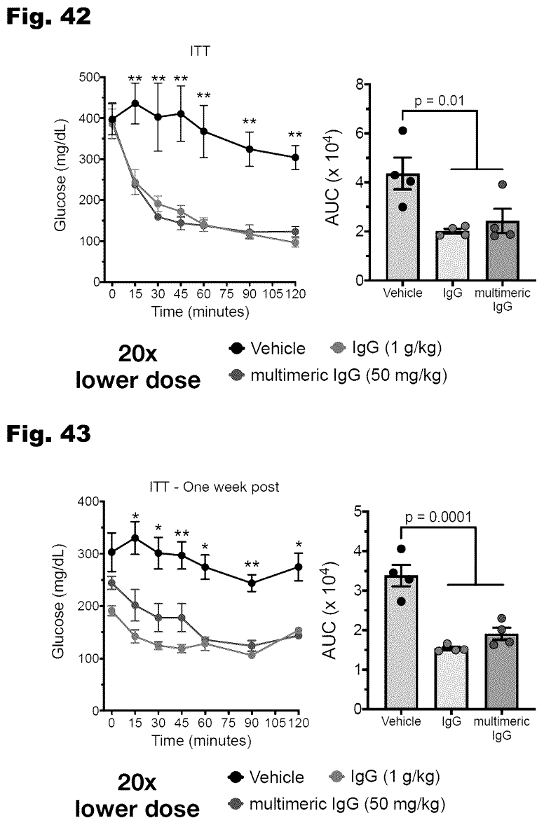

The multimeric IgG Fc peptides were administered to diabetic mice and compared with non-multimeric IgG Fc peptides and vehicle control. IgG (dose 1 g kg⁻¹) or multimeric IgG (dose 50 mg kg⁻¹) was administered by intraperitoneal injection to hyperglycemic 10-week old Lepr^{db/db} and insulin tolerance was monitored over one week. Saline injections were used as a control. Insulin tolerance tests (ITT) were performed on the diabetic mice. Mice treated with multimeric and non-multimeric IgG Fc peptides significantly increased insulin sensitivity as can be seen by the reduced level of circulating glucose (FIG. 42). Further, a single administration of multimeric and non-multimeric IgG Fc peptides maintained the increased insulin sensitivity one week after administration (FIG. 43). Notably, a 20-fold lower dose of multimeric IgG Fc peptides produced similar results to non-multimeric IgG Fc peptides.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. The use of an immunoglobin or an immunoglobin Fc peptide in the manufacture of a medicament for the treatment of a medicament for the treatment of insulin resistance, hyperglycemia, prediabetes, or type 2 diabetes.

2. The manufacture of a medicament of claim 1, wherein the immunoglobin or the immunoglobin Fc peptide comprises an IgG or an IgG Fc peptide.

3. The manufacture of a medicament of claim 2, wherein the IgG or the IgG Fc peptide comprises an IgG1, an IgG2, an IgG3, or an IgG4.

4. (canceled)

5. The manufacture of a medicament of claim 1, wherein the immunoglobin or the immunoglobin Fc peptide is sialylated.

6. The manufacture of a medicament of claim 1, wherein the immunoglobin or the immunoglobin Fc peptide is truncated, modified, chimerized, or conjugated.

7. The manufacture of a medicament of claim 1, wherein the immunoglobin or the immunoglobin Fc peptide comprises a chimeric immunoglobin or a chimeric immunoglobin Fc peptide, wherein the chimeric immunoglobin or the chimeric immunoglobin Fc peptide comprises IaG.

8. (canceled)

9. The manufacture of a medicament of claim 1, wherein the medicament is for improvement of insulin sensitivity.

10. The manufacture of a medicament of claim 1, wherein the medicament is for improvement of glucose tolerance.

11. The manufacture of a medicament of claim 1, wherein the medicament is for improvement of β cell function.

12. The manufacture of a medicament of claim 1, wherein the medicament is for reduction of adipose tissue inflammation.

13. A method of treating insulin resistance, hyperglycemia, prediabetes, or type 2 diabetes, comprising:
administering to a subject a medicament comprising an immunoglobin or an immunoglobin Fc peptide.

14. The method of treatment of claim 13, wherein the immunoglobin or the immunoglobin Fc peptide comprises an IgG or an IgG Fc peptide.

15. The method of treatment of claim 14, wherein the IgG or the IgG Fc peptide comprises an IgG1, an IgG2, an IgG3, or an IgG4.

16. (canceled)

17. The method of treatment of claim 13, wherein the immunoglobin or the immunoglobin Fc peptide is sialylated.

18. The method of treatment of claim 13, wherein the immunoglobin or the immunoglobin Fc peptide is truncated, modified, chimerized, or conjugated.

19. The method of treatment of any one of claims 13-18, wherein the immunoglobin or the immunoglobin Fc peptide comprises a chimeric immunoglobin or a chimeric immunoglobin Fc peptide, wherein the chimeric immunoglobin or the chimeric immunoglobin Fc peptide comprises IgG.

20. (canceled)

21. The method of treatment of claim 13, wherein the medicament is for improvement of insulin sensitivity.

22. The method of treatment of claim 13, wherein the medicament is for improvement of glucose tolerance.

23. The method of treatment of claim 13, wherein the medicament is for improvement of β cell function.

24. The method of treatment of claim 13, wherein the medicament is for reduction of adipose tissue inflammation.

25-70. (canceled)
